(19) United States
(12) Patent Application Publication
(10) Pub. No.: US 2026/0335463 A1
(43) Pub. Date: Oct. 29, 2026

(54) Nucleic Acid Payload Delivery Systems, Compositions, and Methods

(71) Applicant: Seawolf Therapeutics, Inc., San Diego, CA (US)

(72) Inventors: Rob BURKE, Carlsbad, CA (US); Amit SAGI, San Diego, CA (US); Jingjing JIANG, San Diego, CA (US); Xinggang LIU, San Diego, CA (US)

(21) Appl. No.: 19/115,648

(22) PCT Filed: Oct. 25, 2023
(86) PCT No.: PCT/US2023/035933

§ 371 Date: Mar. 26, 2025

Related U.S. Application Data
(60) Provisional application No. 63/419,890, filed on Oct. 27, 2022.
(60) Provisional application No. 63/430,950, filed on Dec. 7, 2022.
(60) Provisional application No. 63/454,505, filed on Mar. 24, 2023.

(51) Int. Cl. A61K 48/00; A61K 9/51; A61K 38/16; A61K 38/18; C12N 15/85; C12N 15/88

(52) U.S. Cl. CPC A61K 48/005; A61K 9/5123; A61K 9/5192; A61K 38/164; A61K 38/1816; A61K 48/0033; A61K 48/0075; C12N 15/85; C12N 15/88

(57) ABSTRACT

Nucleic acid payload delivery systems, and related compositions and kits are provided. Aspects of such systems, compositions, and kits include lipid nanoparticle (LNP)—complexed payload DNAs and auxiliary RNAs. In some instances, the DNA and RNA components are part of the same LNP, while in other instances the DNA and RNA components are part of different LNPs. Also provided are methods of making and using such systems, compositions, and kits for the delivery of LNP complexed nucleic acids into cells, such as cells of a subject in vivo.

### CROSS REFERENCE TO RELATED APPLICATIONS

This application claims the benefit of U.S. Provisional Application No. 63/419,890, filed Oct. 27, 2022; U.S. Provisional Application No. 63/430,950, filed Dec. 7, 2022; and U.S. Provisional Application No. 63/454,505, filed Mar. 24, 2023, the entirety of each of which is hereby incorporated by reference.

### INTRODUCTION

In the developed world where communicable disease mortality is infrequent, genetic disorders, although individually rare, collectively represent a significant disease burden, particularly for children, resulting in substantial disability and mortality. Monogenic diseases are estimated to affect up to 6% of people at some point in their lives. Quantifying the total burden of genetic diseases is difficult and, while many causative loci are known, genetic counseling has only had a minimal impact in reducing overall prevalence by perhaps 5% (see e.g., Blencowe et al. J Community Genet. 2018). Genetic diseases include many rare liver diseases such as phenylketonuria, ornithine transcarbamylase deficiency, arginase-1 deficiency, α-1 antitrypsin deficiency, mucopolysaccharidosis, hemophilia A, hemophilia B, and the like. The large collective burden of genetic diseases, coupled with the low impact genetic counseling has had in reducing that burden, exemplifies the substantial ongoing impact these diseases have on global health.

In the field of gene therapy, viral vectors, such as vectors based on the AAV, are commonly employed to deliver genes to the nucleus. However, AAV vectors are limited in the size of genetic cargo that can be packaged. Accordingly, any genetic cargo greater than 4.7 KB is not suitable for delivery with AAV vectors, which limits the utility of such vectors for many indications. In addition, viral vectors, such as AAV, induce an antibody response, limiting redosing, which is not suitable for some indications. Moreover, in indications where the target cells are dividing, such as the liver, expression from successfully transduced cells can be reduced or lost with cell division and turnover, requiring redosing—which may not be possible or effective due to immune memory. In addition, many subjects have pre-existing immunity to commonly used viral vectors such as AAV, which can limit even initial treatment with an AAV gene therapy. Furthermore, viral vector such as AAV can be toxic at the doses that would be required to achieve therapeutic benefit in some indications.

Lipid nanoparticles provide an alternative to viral gene therapy. While lipid nanoparticles have been developed and employed for delivery of many RNA therapeutics, lipid-nanoparticle-delivered RNA has limited therapeutic longevity. DNA delivered by lipid nanoparticles designed for RNA delivery results in poor efficiency and significant innate immune response activation in treated subjects.

### SUMMARY

Nucleic acid payload delivery systems, and related compositions and kits are provided. Aspects of such systems, compositions, and kits include lipid nanoparticle (LNP)—complexed payload DNAs and auxiliary RNAs. In some instances, the DNA and RNA components are part of the same LNP, while in other instances the DNA and RNA components are part of different LNPs. Also provided are methods of making and using such systems, compositions, and kits for the delivery of LNP-complexed nucleic acids into cells, such as cells of a subject in vivo.

### BRIEF DESCRIPTION OF THE FIGURES

FIG. 1 depicts EPO serum concentrations following administration of EPO DNA LNPs to mice, demonstrating disproportionately low expression from payload DNA with increasing dosages of LNP in vivo.

FIG. 2 provides the EPO serum concentrations in mice following dosing of LNPs formulated with EPO DNA alone; co-dosing of LNPs containing EPO DNA and LNPs containing GFP DNA; or pre-dosed with LNPs containing GFP DNA and then dosed 15 min. later with LNPs containing EPO DNA, and demonstrates that co-administration of stuffer DNA LNP (the GFP DNA LNP) with low dose payload DNA LNP (the EPO DNA LNP) results in levels of payload DNA expression approaching those observed with high dose payload DNA LNP administration alone.

FIG. 3 depicts EPO serum concentrations following administration to mice of LNPs formulated with EPO DNA LNP alone; LNPs co-formulated with EPO DNA and GFP DNA; or LNPs co-formulated with EPO DNA and GFP mRNA and demonstrates that co-formulation of either non-therapeutic DNA LNP or mRNA LNP results in enhanced expression from LNP-delivered DNA payloads.

FIG. 4 depicts cytokine activity following administration to mice of LNPs formulated with EPO DNA alone; LNPs co-formulated with EPO DNA and GFP DNA; or LNPs co-formulated with EPO DNA and GFP mRNA and demonstrates that co-formulation of mRNA LNPs with DNA LNPs r mitigates the immune response to delivered DNA LNPs.

FIG. 5 depicts EPO serum concentrations following administration to mice of LNPs formulated with TetO-containing payload DNA alone or co-formulated with GFP mRNA or TetR mRNA, and demonstrates that co-formulation with mRNA encoding a different protein enhances payload DNA expression, sequence independently, and that use of both TetO-containing payload and TetR-expressing mRNA further enhances expression from payload DNA LNPs.

### DEFINITIONS

The terms “polypeptide,” “polypeptide sequence,” “peptide,” “peptide sequence,” “protein,” “protein sequence” and “amino acid sequence” are used interchangeably herein to designate a linear series of amino acid residues connected one to the other by peptide bonds, which series may include proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, beta-galactosidase, luciferase, and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be taken to mean that such peptide bond or covalent bond to a carboxyl or hydroxyl end group is not present, as it is conventional in representation of amino acid sequences to omit such.

The term “polynucleotide,” “polynucleotide sequence,” “oligonucleotide,” “oligonucleotide sequence,” “oligomer,” “oligo,” “nucleic acid sequence” or “nucleotide sequence” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer having purine and pyrimidine bases or other natural, chemically or biochemically modified, non-natural, or derivatized nucleotide bases.

The terms “derivative” and “variant” refer without limitation to any compound such as nucleic acid or protein that has a structure or sequence derived from the compounds disclosed herein and whose structure or sequence is sufficiently similar to those disclosed herein such that it has the same or similar activities and utilities or, based upon such similarity, would be expected by one skilled in the art to exhibit the same or similar activities and utilities as the referenced compounds, thereby also interchangeably referred to “functionally equivalent” or as “functional equivalents.” Modifications to obtain “derivatives” or “variants” may include, for example, addition, deletion and/or substitution of one or more of the nucleic acids or amino acid residues.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/Selenium-containing | Ser, Cys, Thr, Met |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a preferred predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “complementary” or “substantially complementary,” interchangeably used herein, means that a nucleic acid (e.g., DNA or RNA) has a sequence of nucleotides that enables it to non-covalently bind, i.e., form Watson-Crick base pairs and/or G/U base pairs to another nucleic acid in a sequence-specific, antiparallel, manner (i.e., a nucleic acid specifically binds to a complementary nucleic acid). As is known in the art, standard Watson-Crick base-pairing includes: adenine (A) pairing with thymidine (T), adenine (A) pairing with uracil (U), and guanine (G) pairing with cytosine (C).

A DNA sequence that “encodes” a particular RNA is a DNA nucleic acid sequence that is transcribed into RNA when placed under the control of appropriate regulatory sequences. A DNA polynucleotide may encode an RNA (mRNA) that is translated into protein, or a DNA polynucleotide may encode an RNA that is not translated into protein (e.g., tRNA, rRNA, or a guide RNA or other RNA that does not encode a protein; also called “non-coding” RNA or “ncRNA”). A protein coding sequence or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp/codon/(visited Mar. 20, 2008). By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

The term “recombinant” or “engineered” when used with reference, for example, to a cell, a nucleic acid, a protein, or a vector, indicates that the cell, nucleic acid, protein or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

The term “genomic DNA” or “genomic sequence” refers to the DNA of a genome of an organism including, but not limited to, the DNA of the genome of a bacterium, fungus, archea, plant or animal.

As used herein, “transgene,” “exogenous gene” or “exogenous sequence,” in the context of nucleic acid, refers to a nucleic acid sequence or gene that was not present in the genome of a cell but artificially introduced into the cell and, possibly, the genome of the cell, e.g., via genome-edition.

As used herein, “endogenous gene” or “endogenous sequence,” in the context of nucleic acid, refers to a nucleic acid sequence or gene that is naturally present in the genome of a cell, without being introduced via any artificial means.

The term “expression cassette” refers to a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule having a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. The genetically modified (or transformed or transfected) cells that have therapeutic activity, e.g., treating hemophilia A, can be used and referred to as therapeutic cells.

The term “concentration” used in the context of a molecule such as peptide fragment refers to an amount of molecule, e.g., the number of moles of the molecule, present in a given volume of solution.

The terms “individual,” “subject” and “host” are used interchangeably herein and refer to any subject for whom diagnosis, treatment or therapy is desired. In some aspects, the subject is a mammal. In some aspects, the subject is a human being. In some aspects, the subject is a patient. In some aspects, the subject is a human patient. In some aspects, the subject can have or is suspected of having a disorder or health condition associated with a gene-of-interest (GOI). In some aspects, the subject is a human who is diagnosed with a risk of disorder or health condition associated with a GOI at the time of diagnosis or later. In some cases, the diagnosis with a risk of disorder or health condition associated with a GOI can be determined based on the presence of one or more mutations in the endogenous GOI or genomic sequence near the GOI in the genome that may affect the expression of GOI.

The term “treatment” referring to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition (e.g., hemophilia A) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

The terms “effective amount,” “pharmaceutically effective amount,” or “therapeutically effective amount” as used herein mean a sufficient amount of the composition to provide the desired utility when administered to a subject having a particular condition. The term “therapeutically effective amount” therefore refers to an amount of therapeutic cells or a composition having therapeutic cells that is sufficient to promote a particular effect when administered to a subject in need of treatment. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The term “pharmaceutically acceptable excipient” as used herein refers to any suitable substance that provides a pharmaceutically acceptable carrier, additive or diluent for administration of a compound(s) of interest to a subject. “Pharmaceutically acceptable excipient” can encompass substances referred to as pharmaceutically acceptable diluents, pharmaceutically acceptable additives, and pharmaceutically acceptable carriers.

A “biological sample” encompasses a variety of sample types obtained from an individual and can be used in a testing, diagnostic, or monitoring assay. The definition encompasses blood, and components thereof (e.g., serum, plasma, etc.), and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

### DETAILED DESCRIPTION

Nucleic acid payload delivery systems, and related compositions and kits are provided. Aspects of such systems, compositions, and kits include lipid nanoparticle (LNP)-complexed payload DNAs and auxiliary RNAs. In some instances, the DNA and RNA components are part of the same LNP, while in other instances the DNA and RNA components are part of different LNPs. Also provided are methods of making and using such systems, compositions, and kits for the delivery of LNP-complexed nucleic acids into cells, such as cells of a subject in vivo.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Systems & Compositions

### Lipid Nanoparticle (LNP) Systems

As summarized above, the present disclosure includes nucleic acid payload delivery systems and compositions, which include LNP systems and compositions that include LNP-complexed payload DNAs and auxiliary RNAs. In some instances, the DNA and RNA components are complexed together in the same LNP. In other words, they are co-formulated, or encapsulated, in the same LNP. In some instances, the DNA and RNA components are complexed in separate LNPs. In other words, they are formulated, or encapsulated, in separate LNPs. The described LNP systems and compositions are useful for the delivery of payload DNA into desired cells, including cells of a subject in vivo. As will be recognized by the relevant ordinarily skilled artisan, description of particular systems provided herein will, for brevity, readily apply generally to compositions and description of particular compositions provided herein will, for brevity, readily apply generally to systems, whether or not the description specifically refers to systems and compositions.

The LNP systems of the present disclosure include where both a payload DNA and an auxiliary RNA are encapsulated in the same LNP, as well as where the payload DNA and the auxiliary RNA are encapsulated in separate LNPs. Where separate LNPs are employed, the LNPs employed to separately encapsulate the payload DNA and the auxiliary RNA may be of the same or different compositions. Aspects of the present disclosure involve the co-delivery or co-timely or coincidental administration of the payload DNA and auxiliary RNA in an LNP system, either through the use of a single LNP that includes both the payload DNA and auxiliary RNA or by co-administration of a payload DNA LNP and an auxiliary RNA LNP of an LNP system as described.

The LNP systems of the present disclosure may, in some instances, provide for certain advantage, e.g., as compared to conventional LNP systems. For example, the LNP systems find certain advantageous use in the administration of payload DNAs in vivo to subjects, including subjects in need of the payload DNA and/or an expression product of the payload DNA. Accordingly, the systems, compositions, kits, and methods of the present disclosure provide for enhanced functionality, such as e.g., enhanced delivery of nucleic acid components, enhanced expression from payload DNAs containing coding sequences, reduced immune responses in subjects administered the compositions, and combinations thereof. Such enhanced functionality in vivo provides for overall enhanced therapeutic efficacy of LNP system.

Systems and compositions of the present disclosure generally include one or more LNP components, including e.g., where the nucleic acid components of the system or composition are encapsulated, either together or separately, in such LNP component(s). Useful LNP compositions will vary where certain exemplary compositions may include combinations of one or more ionizable lipids, one or more co-lipids/helper lipids, cholesterol or cholesterol analogs, polyethylene glycol (PEG) or PEG lipids, and other components, such as targeting components and condensing agents, where some of the components listed may be optionally present. A relevant artisan will readily understand the LNP compositions may vary widely.

In some embodiments, LNPs of the present disclosure may be composed of at least payload DNA and/or an auxiliary RNA molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol). For example, lipid nanoparticles may comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5 (3): 498-507.

In some embodiments, a lipid nanoparticle has a mean diameter between about 10 and about 1000 nm or between about 10 and 500 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm, including e.g., between about 25 and about 150 nm or between about 25 and about 100 nm. In some embodiments, a lipid nanoparticle preparation (e.g., composition comprising a plurality of lipid nanoparticles) has a size distribution in which the mean size (e.g., diameter) is about 50 nm to about 200 nm, about 50 nm to about 150 nm, about 50 nm to about 100 nm, 60 nm to about 200 nm, about 60 nm to about 150 nm, about 60 nm to about 100 nm, about 70 nm to about 200, or about 70 nm to about 150 nm, and more typically the mean size is about 100 nm or less, about 90 nm or less, about 80 nm or less, or about 70 nm or less.

Useful LNPs may include an ionizable lipid. The ionizable lipid is typically employed to condense a nucleic acid cargo at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein. Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, U52013/0116307, US2010/0062967, US2013/0202684, US2014/0141070, US2014/0255472, US2014/0039032, US2018/0028664, U52016/0317458, and US2013/0195920.

Various LNP formulations known in the art can be used to deliver the nucleic acid components of the systems and compositions as described herein. For example, various LNP formulations and delivery methods using lipid nanoparticles are described in U.S. Pat. Nos. 9,404,127, 9,006,417, 9,518,272, and U.S. Patent Application No. 63/415,229. Such particles can be prepared by high energy mixing of ethanolic lipids with aqueous nucleic acid at low pH which protonates the ionizable lipid and provides favorable energetics for nucleic acid/lipid association and nucleation of particles. The particles can be further stabilized through aqueous dilution and removal of the organic solvent. The particles can be concentrated to the desired level.

### Nucleic Acid Components

As summarized above, the systems of the present disclosure include nucleic acid components including payload DNAs and auxiliary RNAs. While payload DNA and auxiliary RNA are components of the described LNP systems, such nucleic acid components can, but need not necessarily be, complexed together in the same LNP. In some instances, there may be practical advantages to co-encapsulation of payload DNAs and auxiliary RNAs. In some instances, there may be practical advantages to payload DNAs and auxiliary RNAs being separately complexed with separate LNPs.

Payload DNA, as described in more detail below, generally provides for a function, including a function needed by the subject to which the LNP system is delivered. Auxiliary RNA, as described in more detail below, generally provides a supportive function in the delivery and/or expression of the payload DNA within the LNP system. For example, inclusion of the auxiliary RNA in the LNP system may result in enhanced delivery of the payload DNA to cells (e.g., as compared to the delivery resulting from a corresponding system that does not include the auxiliary RNA), e.g., 2-fold or more, such as 5-fold of more, including 10-fold or more; inclusion of the auxiliary RNA in the LNP system may result in enhanced expression of a coding sequence present in the payload DNA (e.g., as compared to the level of expression resulting from a corresponding system that does not include the auxiliary RNA); inclusion of the auxiliary RNA in the LNP system may result in enhanced incorporation of the payload DNA into a targeted locus of the genome of the target cell (e.g., as compared to the level of incorporation resulting from a corresponding system that does not include the auxiliary RNA); or some combination of multiple enhancements. In some instances, inclusion of the auxiliary RNA in the LNP system may reduce the occurrence of an undesired biological result due to delivery of the payload DNA (e.g., as compared to the level of the undesired result occurring from use of a corresponding system that does not include the auxiliary RNA). For example, inclusion of the auxiliary RNA in the LNP system may result in a reduction of an immune reaction in the subject to which the LNP containing the payload DNA is delivered (e.g., as compared to the level of immune response resulting from delivery of a corresponding system that does not include the auxiliary RNA). In some instances, the system may provide for both one or more enhancements and one or more reductions in an undesired outcome.

The term “payload DNA” generally refers to a DNA molecule that is delivered to a cell, generally a cell of a subject delivered in vivo, to provide a function, e.g., to provide a therapeutic outcome for the subject. The functions provided by delivered payload DNA will vary and may be related to a therapeutic need of a subject (i.e., a subject in need of receiving a particular payload DNA). Useful nonlimiting examples of functions of payload DNAs include: therapeutic functions (including e.g., providing a corrected copy of a gene, providing a hyperfunctional a gene or gene product, providing a synthetic gene or gene product, etc.), research functions, diagnostic functions, etc.

In some instances, a cargo nucleic acid may be employed as a payload DNA. Useful cargo nucleic acids will vary. In some instances, the cargo nucleic acid is 500 nt or more, for example 1 kb, 2 kb, 3 kb, 4 kb, or 5 kb or more, e.g. 6 kb, 7 kb, 8 kb, 9 kb, 10 kb or more, in some cases, greater than 4.7 kb or 15 KB or more. The cargo nucleic may have any desired sequence. In some instances, the cargo nucleic acid may include one or more of coding sequences, promoters, sequences homologous to the genomic DNA of the targeted nucleus (e.g., to provide for genomic integration of the cargo nucleic acid), untranslated sequences (5′ UTR, 3′ UTR), polyadenylation sequences, and the like.

In some instances, a nuclear targeted deoxyribonucleic acid (NTDNA) may be employed as a payload DNA, where a NTDNA includes a DNA nuclear targeting sequence (DTS) and a cargo nucleic acid heterologous to the DTS. By “heterologous” to the DTS is meant that the cargo nucleic acid is not naturally associated with the DTS, e.g., is not part of the same gene as the DTS in nature. A DTS refers to a nucleotide sequence that mediates the translocation of a polynucleotide that comprises it into the nucleus of a cell. Put another way, a DTS will include one or more sequences that promotes the translocation of the payload DNA, e.g., a cargo nucleic acid when a NTDNA is employed, into the nucleus of a eukaryotic cell. Useful DTSs, and useful components thereof (including but not limited to e.g., targeting factor binding sequences (TFBS) for a nuclear targeting factor (NTF) and components thereof, are further described herein. In some embodiments, as described herein, an auxiliary RNA may encode for an NTF or a portion thereof.

The form or structure of payload DNA employed in the described systems will vary. Accordingly useful payload DNA include linear, circular, single-stranded, and double-stranded DNA configurations, including but not limited to e.g., plasmid DNA (pDNA), nanoplasmid DNA (npDNA), mini-circle DNA (mcDNA), single-stranded circular DNA (circDNA), doggy-bone DNA (dbDNA), DNA comprising terminal repeat sequences (e.g. closed ended DNA (ceDNA)), and 3D DNA.

As such, payload DNAs may, in some instances, include a coding sequence, which may encode for one or multiple encoded products. Useful encoded products include but are not limited to e.g., noncoding nucleic acids and nucleic acids coding for one or more proteins and/or peptides, including but not limited to e.g., one or more of the therapeutic proteins and/or peptides described herein. In some embodiments, a payload may include nucleic acid sequence coding for an enzyme, such as e.g., a nuclease, a DNA base editor, an RNA editor, or the like. In some embodiments, a payload may include, alone or with other payload elements, a noncoding nucleic acid such as e.g., a microRNA (i.e., miRNA), shRNA, siRNA, piRNA, snoRNA, snRNA, exRNA, scaRNA, lncRNA, guide RNA (gRNA, sgRNA, etc.), or the like. A payload may encode for one encoded product or multiple encoded products, e.g., a payload may encode one polypeptide or multiple polypeptides, such as e.g., 2, 3, 4, 5, 6, 7, 8, or 9 or more polypeptides. Therapeutic products encoded from a payload DNA may be active intrinsically (e.g., within the cell in which the product is encoded) or extrinsically (e.g., outside the cell in which the product is encoded, e.g., in the extracellular space, blood, plasma, on the surface of another cell, inside another cell, etc.). An encoded therapeutic that is active extrinsically may include an element that facilitates export of the therapeutic extracellularly, such as e.g., a secretory signal peptide.

In some embodiments, an encoded polypeptide is a therapeutic polypeptide that includes, or consists of, a therapeutic protein or peptide. Useful therapeutic proteins and peptides include, but are not limited to, e.g., secreted factors, hormones, chemokines, cytokines, transcription factors, ligands for receptors, receptor-blocking proteins and peptides, enzymes, extracellular matrix proteins, signaling proteins, antibodies, and the like. In certain embodiments, the therapeutic protein comprises a functional protein lacking (i.e., deficient and/or absent) in a liver disease or a disease manifesting from a deficiency in a protein produced primarily in the liver.

Non-limiting examples of therapeutic proteins and peptides include Lepirudin, Cetuximab, Dornase alfa, Denileukin diftitox, Etanercept, Bivalirudin, Leuprolide, Peginterferon alfa-2a, Alteplase, Interferon alfa-n1, Darbepoetin alfa, Reteplase, Erythropoietin (EPO), Epoetin alfa, Salmon Calcitonin, Interferon alfa-n3, Pegfilgrastim, Sargramostim, Secretin, Peginterferon alfa-2b, Asparaginase, Thyrotropin Alfa, Antihemophilic Factor, Anakinra, Gramicidin D, Intravenous Immunoglobulin, Anistreplase, Insulin Regular, Tenecteplase, Menotropins, Interferon gamma-1b, Interferon Alfa-2a, Recombinant, Coagulation factor VIIa, Oprelvekin, Palifermin, Glucagon recombinant, Aldesleukin, Botulinum Toxin Type B, Omalizumab, Lutropin alfa, Insulin Lispro, Insulin Glargine, Collagenase, Rasburicase, Adalimumab, Imiglucerase, Abciximab, Alpha-1-proteinase inhibitor, Pegaspargase, Interferon beta-1a, Pegademase bovine, Human Serum Albumin, Eptifibatide, Serum albumin iodonated, Infliximab, Follitropin beta, Vasopressin, Interferon beta-1b, Interferon alfacon-1, Hyaluronidase, Insulin (porcine), Trastuzumab, Rituximab, Basiliximab, Muromonab, Digoxin Immune Fab (Ovine), Ibritumomab, Daptomycin, Tositumomab, Pegvisomant, Botulinum Toxin Type A, Pancrelipase, Streptokinase, Alemtuzumab, Alglucerase, Capromab, Laronidase, Urofollitropin, Efalizumab, Serum albumin, Choriogonadotropin alfa, Antithymocyte globulin, Filgrastim, Coagulation factor IX, Becaplermin, Agalsidase beta, Interferon alfa-2b, Oxytocin, Enfuvirtide, Palivizumab, Daclizumab, Bevacizumab, Arcitumomab, Eculizumab, Panitumumab, Ranibizumab, Idursulfase, Alglucosidase alfa, Exenatide, Mecasermin, Pramlintide, Galsulfase, Abatacept, Cosyntropin, Corticotropin, Insulin aspart, Insulin detemir, Insulin glulisine, Pegaptanib, Nesiritide, Thymalfasin, Defibrotide, Natural alpha interferon OR multiferon, Glatiramer acetate, Preotact, Teicoplanin, Canakinumab, Ipilimumab, Sulodexide, Tocilizumab, Teriparatide, Pertuzumab, Rilonacept, Denosumab, Liraglutide, Golimumab, Belatacept, Buserelin, Velaglucerase alfa, Tesamorelin, Brentuximab vedotin, Taliglucerase alfa, Belimumab, Aflibercept, Asparaginase Erwinia chrysanthemi, Ocriplasmin, Glucarpidase, Teduglutide, Raxibacumab, Certolizumab pegol, Insulin (isophane), Epoetin zeta, Obinutuzumab, Fibrinolysin aka plasmin, Follitropin alpha, Romiplostim, Lucinactant, Natalizumab, Aliskiren, Secukinumab, Somatotropin Recombinant, Drotrecogin alfa, Alefacept, OspA lipoprotein, Urokinase, Abarelix, Sermorelin, Aprotinin, Gemtuzumab ozogamicin, Satumomab Pendetide, Albiglutide, Alirocumab, Ancestim, Antithrombin Alfa, Antithrombin III human, Asfotase Alfa, Atezolizumab, Beractant, Blinatumomab, C1 Esterase Inhibitor (Human), Coagulation Factor XIII A-Subunit (Recombinant), Conestat alfa, Daratumumab, Desirudin, Dulaglutide, Elosulfase alfa, Elotuzumab, Evolocumab, Fibrinogen (Human), Filgrastim-sndz, Gastric intrinsic factor, Hepatitis B immune globulin, Human calcitonin, Human Clostridium Tetani toxoid immune globulin, Human rabies virus immune globulin, Human Rho (D) immune globulin, Hyaluronidase (Human Recombinant), Idarucizumab, Immune Globulin Human, Vedolizumab, Ustekinumab, Turoctocog alfa, Simoctocog Alfa, Siltuximab, Sebelipase alfa, Sacrosidase, Ramucirumab, Prothrombin, Poractant alfa, Pembrolizumab, Peginterferon beta-1a, Ofatumumab, Obiltoxaximab, Nivolumab, Necitumumab, Metreleptin, Methoxy polyethylene glycol-epoetin beta, Mepolizumab, Ixekizumab, Insulin (pig), Insulin Degludec, Insulin (bovine), Thyroglobulin, Anthrax immune globulin human, Anti-inhibitor coagulant complex, Anti-thymocyte Globulin (Equine), Anti-thymocyte Globulin (Rabbit), Brodalumab, C1 Esterase Inhibitor (Recombinant), Canakinumab, Chorionic Gonadotropin (Human), Chorionic Gonadotropin (Recombinant), Coagulation factor X human, Dinutuximab, Efmoroctocog alfa, Factor IX (Human), Hepatitis A Vaccine, Human Varicella-Zoster Immune Globulin, Ibritumomab tiuxetan, Lenograstim, Pegloticase, Protamine sulfate, Protein S human, Sipuleucel-T, Somatropin recombinant, Susoctocog alfa, Thrombomodulin Alfa, and the like. In some instances, useful therapeutic proteins include proteins that specifically bind a target protein where such binding results in a therapeutic effect, such as therapeutic antibodies which bind a target protein to produce a therapeutic effect. Non-limiting examples of therapeutic antibodies include 9E10, 8H9, Abagovomab, Abatacept, Abciximab, Abituzumab, Abrilumab, Actoxumab, Adalimumab, Adecatumumab, Aducanumab, Afelimomab, Afutuzumab, Alacizumab pegol, ALD518, Alefacept, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atacicept, Atezolizumab, Atinumab, Atlizumab/tocilizumab, Atorolimumab, AVE1642, Bapineuzumab, Basiliximab, Bavituximab, Bectumomab, Begelomab, Belimumab, Benralizumab, Bertilimumab, Besilesomab, Bevacizumab, Bevacizumab/Ranibizumab, Bezlotoxumab, Biciromab, Bimagrumab, Bimekizumab, Bivatuzumab mertansine, Blinatumomab, Blosozumab, BMS-936559, Bococizumab, Brentuximab, Brentuximabvedotin, Briakinumab, Brodalumab, Brolucizumab, Brontictuzumab, Canakinumab, Cantuzumab mertansine, Cantuzumab ravtansine, Caplacizumab, Capromab pendetide, Carlumab, Catumaxomab, cBR96-doxorubicin immunoconjugate, CDP791, Cedelizumab, Certolizumab, Cetuximab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CP-751871, CR6261, Crenezumab, CS-1008, Dacetuzumab, Daclizumab, Dalotuzumab, Dapirolizumab pegol, Daratumumab, Dectrekumab, Demcizumab, Denintuzumab mafodotin, Denosumab, Derlotuximab biotin, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Epratuzumab, Erlizumab, Ertumaxomab, Etanercept, Etaracizumab, Etrolizumab, Evinacumab, Evolocumab, Exbivirumab, F19, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab, Gevokizumab, Girentuximab, Glembatumumab vedotin, Golimumab, Gomiliximab, Guselkumab, HGS-ETR2, Ibalizumab, Ibritumomab, Icrucumab, Idarucizumab, Igovomab, IIIA4, IM-2C6, IMAB362, Imalumab, IMC-A12, Imciromab, Imgatuzumab, Inclacumab, Indatuximab ravtansine, Indusatumab vedotin, Infliximab, Inolimomab, Inotuzumab ozogamicin, Intetumumab, Ipilimumab, Iratumumab, Isatuximab, Itolizumab, Ixekizumab, KB004, Keliximab, Labetuzumab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Matuzumab, Mavrilimumab, MEDI4736, Mepolizumab, Metelimumab, Milatuzumab, Minretumomab, Mirvetuximab soravtansine, Mitumomab, MK-0646, Mogamulizumab, Morolimumab, Morolimumab immune, Motavizumab, Moxetumomab pasudotox, MPDL33280A, Muromonab-CD3, Nacolomab tafenatox, Namilumab, Naptumomab estafenatox, Narnatumab, Natalizumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, R1507, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Rituximab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teplizumab, Teprotumumab, Tesidolumab, Tetulomab, TGN1412, Ticilimumab/tremelimumab, Tigatuzumab, Tildrakizumab, TNX-650, Tocilizumab, Toralizumab, Tosatoxumab, Tovetumab, Tralokinumab, Trastuzumab, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, Zolimomab aritox, and the like.

Useful payload DNAs may include coding sequence for treating a monogenic condition including, but are not limited to e.g., coding sequence encoding full-length, partial, and modified forms of Copper-transporting ATPase 2 (ATP7B), Hereditary hemochromatosis protein (HFE), Hemojuvelin, Hepcidin (HAMP), Transferrin receptor protein 2 (TFR2), Solute carrier family 40 member 1 (SLC40A1), Factor IX, Factor VIII, von Willebrand factor, Carbamoyl-phosphate synthase (CPS1), N-acetylglutamate synthase (NAGS), Ornithine transcarbamylase (OTC), alpha-galactosidase A gene (GLA), phenylalanine hydroxylase enzyme (PAH), arginase (ARG, including ARG1), alpha-1 antitrypsin (AAT), fumarylacetoacetate hydrolase (FAH), Argininosuccinate lyase (ASL), Argininosuccinate synthase (ASS, including ASS1), Ornithine translocase (ORNT1), citrin, UDP-glucuronosyltransferase 1A1 (UGT1A1), Transthyretin (TTR), Serine—pyruvate aminotransferase (AGXT), Complement factor H (CFH), the like, and combinations thereof. As will be readily understood, delivered coding sequences may be employed for expression outside the genome of a cell (e.g., episomally) or from within the genome of a cell (e.g., following integration of the coding sequence into a chromosome, e.g., at an endogenous or exogenous locus).

In some instances, a payload DNA includes or consists of an expression cassette, e.g., which may include all the elements necessary for transcription of a coding sequence from the payload DNA.

In some instances, while a payload DNA may code for a particular amino acid sequence, the payload DNA may not include all the elements necessary for transcription (i.e., may exclude at least one sequence element necessary for transcription and expression) and thus no product is expressed from the payload DNA. Such encoding-but-non-expressing payload DNAs may find various uses, such as e.g., as a donor template for DNA replacement, gene correction, gene modification, etc. In some instances, an expression product is not produced from a payload DNA. In some instances, an expression product is produced from a payload DNA after integration of the payload DNA into the genome of the cell. In some instances, an expression product is not produced from a payload DNA until the payload DNA is integrated into the genome of the cell.

In some instances, a payload DNA does not encode an RNA or polypeptide. In some instances, a payload DNA may partially include or be entirely made of non-coding DNA sequence. In some instances, a payload DNA may provide a donor template, where donor templates may include non-coding DNA, including where a DNA template is entirely non-coding or only a portion of the donor template is non-coding DNA. Accordingly, in some instances, donor templates are useful for correction or replacement of non-coding regions of the genome of a cell. Donor templates, and use of payload DNA to provide a donor template, are described in more detail elsewhere herein.

Useful payloads may include elements for editing of a target locus. For example, a payload may include an exogenous donor template nucleic acid that includes regions of homology (“homology regions”) to a target site flanking a sequence that contains the desired edit. Gene editing payload may repair or otherwise introduce a desired edit at a target locus by various mechanism, including e.g., homology directed repair (HDR).

Homology regions targeted to a genomic locus, sometimes referred to as “homology arms” or separately as a “5′ homology arm” and a “3′ homology arm”, share homology to endogenous nucleic acid 5′ and 3′, respectively, of the target site. Homology arms may vary and may range in size from 200 nt or less to 2000 nt or more, including but not limited to e.g., 200 nt or more, 500 nt or more, 500 nt to 1000 nt, etc. Essentially any nucleic acid edit may be introduced and useful edits may include, a single nucleotide edit (i.e., a change of one base for another, e.g., an A to C, an A to T, an A to G, a C to A, a C to G, a C to T, a G to A, a G to C, a G to T, a T to A, a T to C, or a T to G base change), a change of two or more nucleotides (i.e., a change of a base for another at two or more sites), a single nucleotide insertion, an insertion of two or more nucleotides, a single codon insertion (i.e., an insertion of three nucleotides), an insertion of two or more codons, an insertion of a heterologous coding sequence, a single nucleotide deletion, a deletion of two or more nucleotides, a deletion of one or more codons, a deletion of one or more exons, a deletion of a coding region or a portion thereof, a deletion of a noncoding region or a portion thereof, a gene replacement, a replacements of a portion of a gene, etc.

Elements of a gene editing payload will vary and may include a variety of other elements, including but not limited to e.g., one or more guide RNAs (e.g., gRNA, sgRNA, etc.), nucleases (e.g., Cas nucleases (e.g., Cas9, Cas12, etc.), zinc finger nucleases (ZFN), transcription activator-like effector nucleases (TALENs), meganucleases, etc.) or sequence encoding one or more nucleases, nickases, etc. or sequence encoding a base-editing enzyme (e.g., cytosine base editors, adenine base editors, dual-deaminase editors, etc.) or sequence encoding more than one base-editing enzymes, and the like.

Payload DNA may vary in length, ranging in some instances from 15nt to 15,000 nt, such as 100 to 10,000 nt and including 100 to 5000nt. In some instances, the payload DNA is at least 100 nt in length, including e.g., at least 125 nt in length, at least 150 nt in length, at least 200 nt in length, at least 250 nt in length, at least 300 nt in length, at least 350 nt in length, at least 400 nt in length, at least 450 nt in length, or at least 500 nt in length; including e.g., where such payload DNA is less than 15,000 nt in length, such as 10,000 nt in length or less, 8,000 nt in length or less, 6,000 nt in length or less, 5,000 nt in length or less, 4,000 nt in length or less, 3,000 nt in length or less, 2,500 nt in length or less, 2,000 nt in length or less, 1,500 nt in length or less, or 1,000 nt in length or less.

The payload DNA may vary as desired. In some instances, the payload DNA is 500 nt or more, for example 1 kb, 2 kb, 3 kb, 4 kb, or 5 kb or more, e.g., 6 kb, 7 kb, 8 kb, 9 kb, 10 kb or more, in some cases, 15 kB or more. The payload DNA may have any desired sequence. In some instances, the payload DNA may include one or more of coding sequences, promoters, sequences homologous to the genomic DNA of the targeted nucleus (e.g., to provide for genomic integration of the payload DNA or donor template), untranslated sequences (5′ UTR, 3′ UTR), polyadenylation sequences, and the like.

A payload DNA to be delivered to the nucleus may be configured to be maintained episomally or integrated into the genome, as desired. As such, in some instances a payload DNA is configured to be maintained episomally in the nucleus of a target cell, such that it is not genomically integrated. In other instances, the payload DNA may be configured to be integrated into the genome of a target cell. In such instances, integration may be accomplished using any convenience protocol, such as by using a gene editing system, e.g., as described in greater detail below.

In some instances, the payload DNA includes a coding sequence that may encode essentially any encoded product. By a coding sequence it is meant a nucleic acid sequence that encodes for any expression product, e.g., micro RNA (miRNA), small hairpin RNA (shRNA), circular RNA (circRNA), long noncoding RNA (lncRNA), mRNA, peptide, polypeptide, protein. Where desired, a given coding sequence can encode multiple gene products, e.g., separated by IRES sequence, 2A sequence (e.g., a P2A, a T2A, etc.), or the like. For example, a coding sequence to be delivered by a payload DNA can be configured so that it can be integrated into the genome in operable linkage with its native promoter, for example to replace a mutant coding sequence or to be in operable linkage with an active promoter at a safe harbor (in which instances, the payload DNA need not include, and in some instances does not include, a promoter).

In some instances, nucleic acids of the present disclosure may include one or more components of an expression cassette. By an expression cassette it is meant a nucleic acid sequence comprising a regulatory sequence that includes a promoter operably linked to a coding sequence. In some instances, a nucleic acid may include all elements of an expression cassette necessary for expression from a coding sequence of the nucleic acid. Useful elements of expression cassettes will vary and will generally include a coding sequence (e.g., transgene, a gene of interest (GOI), open reading frame (ORF), etc.) and one or more regulatory elements, where useful regulatory elements may include e.g., promoters, enhancers, untranslated regions (UTRs, inc. 3′UTRs and 5′UTRs), terminators, etc.

In some instances, nucleic acids of the present disclosure may include a promoter. As used herein, the term “promoter” refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes. A promoter sequence may be bounded at its 3′ terminus by the transcription initiation site and extends upstream (5′ direction) to include the minimum number of bases or elements necessary to initiate transcription at levels detectable above background. Useful promoters include, for example, constitutively active promoters, such as the CMV promoter, CAG promoter (which combines the CMV enhancer with the chicken β-actin (CBA) promoter), β-actin promoter, SV-40 promoter, 4×GRM6-SV40, hTTR, hAAT, 3x-Serpina, ubiquitin B/C, EF1-Alpha, EFS, and HBV promoter, etc. Useful promoters also include promoters having more cell-type specific expression patterns, for example for hepatocytes, may include, without limitation the TTR, hAAT (and derivatives), 3xSerpina-TTR, HBV, UbiC, and P3-hybrid promoter. A payload DNA may include a promoter sequence to be delivered to the nucleus so that it can be integrated into the genome, for example to replace a mutant promoter.

In some instances, the payload DNA includes an expression cassette. In some instances, the expression cassette may also comprise one or more nucleic acid sequences including a 5′ UTR, 3′ UTR, polyA tail, or other regulatory element. In embodiments, the expression cassette may include a transgene and one or more regulatory sequences that allows and/or controls the expression of the transgene, e.g., where the expression cassette can include one or more of, e.g., in this order: an enhancer/promoter, an ORF (transgene), a post-transcription regulatory element (e.g., WPRE), and a polyadenylation and termination signal (e.g., BGH polyA). The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer.

As desired, an expression cassette can comprise 4000 or more nucleotides, 5000 or more nucleotides, 10,000 or more nucleotides or 20,000 or more nucleotides, or 30,000 or more nucleotides, or 40,000 or more nucleotides or 50,000 or more nucleotides, and in some instances may range between 4000-10,000 nucleotides or 10,000-50,000 nucleotides, or more than 50,000 nucleotides. The expression cassette to be delivered to the nucleus may be configured so that it can be maintained episomally. Alternatively, an expression cassette to be delivered to the nucleus may be configured so that it can be integrated into the genome.

The coding sequence e.g., transgene, of the expression cassette may vary. In some embodiments, the expression cassette can include a transgene in the range of 500 to 50,000 nucleotides in length. In some embodiments, the expression cassette can include a transgene in the range of 500 to 75,000 nucleotides in length. In some embodiments, the expression cassette can include a transgene which is in the range of 500 to 10,000 nucleotides in length. In some embodiments, the expression cassette can include a transgene which is in the range of 1000 to 10,000 nucleotides in length. In some embodiments, the expression cassette can include a transgene which is in the range of 500 to 5,000 nucleotides in length.

The constructs of embodiments of the herein described systems and compositions do not have the size limitations of encapsulated AAV vectors, and thus enable delivery of large-size expression cassettes to provide efficient expression from large-size transgenes. A given expression cassette can include, for example, an expressible exogenous sequence (e.g., ORF) or transgene that encodes a protein that is either absent, inactive, or of insufficient activity in the recipient subject or a gene that encodes a protein having a desired biological or a therapeutic effect. The transgene can encode a gene product that can function to correct the expression of a defective gene or transcript. In principle, an expression cassette including any gene that encodes a protein, polypeptide or RNA that is either reduced or absent due to a mutation or which conveys a therapeutic benefit when overexpressed is considered to be within the scope of the disclosure.

The expression cassette can include any transgene useful for treating a disease or disorder in a subject. Systems and compositions, e.g., as described herein, can be used to deliver and express any expression product in a subject, where such expression products include, but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences, HBV virus sequences, and the like.

In some instances, an encoded expression product (e.g., as disclosed herein) is used for therapeutic purposes (e.g., for medical, diagnostic, or veterinary uses). In certain embodiments, a payload DNA is useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, and their antisense counterparts (e.g., antagoMiR)), antibodies, antigen binding fragments, or any combination thereof. As such, expression cassettes can encode polypeptides, sense or antisense oligonucleotides, or RNAs (coding or non-coding; e.g., siRNAs, shRNAs, micro-RNAs, and their antisense counterparts (e.g., antagoMiR)).

In some instances, an encoded expression product, e.g., as encoded from a payload DNA or an auxiliary RNA, may is a non-therapeutic expression product (e.g., a non-therapeutic polypeptide, a non-therapeutic RNA, etc.). In some embodiments, an auxiliary RNA encodes only non-therapeutic expression products, e.g., one or more non-therapeutic polypeptides. Non-therapeutic expression products will vary and can include an exogenous sequence that encodes essentially any non-therapeutic protein, such as e.g., a reporter protein (i.e., a “reporter”). Expressed non-therapeutic proteins, e.g., reporter proteins, may or may not have a non-therapeutic function or purpose in a system of the present disclosure. For example, an expressed reporter protein may or may not be assayed or detected. For example, a reporter may be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art. In some instances, although a nucleic acid, such as an auxiliary RNA, encodes a reporter, the reporter may not be employed in methods related to use of the system for any diagnostic or detection purposes. In some instances, non-therapeutic expression products expressed from nucleic acids of the present disclosure, such as an auxiliary RNA, may be employed to provide one or more functions that enhance expression from or delivery of a payload DNA of the system. For example, an auxiliary RNA may be employed that encodes an non-therapeutic expression product that includes a polypeptide with a DNA binding domain (DBD), where the payload DNA comprises a sequence to which the DNA binding domain binds. Such configurations may employ encoded non-therapeutic products to enhance delivery of the payload DNA into the nucleus of the cells, thereby enhancing expression of one or more coding sequences present on the payload DNA.

Coding sequences can be codon optimized for the target host cell. As used herein, the term “codon optimized” or “codon optimization” refers to the process of modifying a nucleic acid sequence for enhanced expression in the cells of the vertebrate of interest, e.g., mouse, NHP, or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein.

In some embodiments, a transgene expressed from a payload DNA is a therapeutic gene. In some embodiments, a therapeutic gene is an antibody, or antibody fragment, or antigen-binding fragment thereof, e.g., a neutralizing antibody or antibody fragment and the like. In some instances, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Of interest in certain embodiments are transgenes that are heterologous relative to one or more other sequences present on the payload DNA. For example, a coding sequence to be delivered to the nucleus so that it can be integrated into the genome in operable linkage with its native promoter, for example to replace a mutant coding sequence or to be in operable linkage with an active promoter at a safe harbor (in which instances, the polynucleotide does not comprise a promoter); or the coding sequence of an expression cassette to be delivered to the nucleus to be either maintained episomally or integrated into the genome. By a coding sequence it is meant a nucleic acid sequence that encodes for any gene product, e.g., micro RNA (miRNA), small hairpin RNA (shRNA), circular RNA (circRNA), long noncoding RNA (lncRNA), mRNA, peptide, polypeptide, protein. A coding sequence can encode multiple gene products, e.g., separated by IRES sequence, 2A sequence, or the like.

Where desired, a given payload DNA, such as a payload DNA including or consisting of a donor template as described herein, may include one or more elements for integration of the payload DNA, or a portion thereof into a target cell genome, such as e.g., flanking sequences that are homologous to genomic regions of the cell, e.g., to promote genomic integration of a donor template. When present, such sequences may vary in length, ranging in some instances from 30 to 5,000 nt, such as 50 to 1000 nt, 100 to 1000 nt, 150 to 1000 nt, 200 to 1000 nt, etc. While the sequences of such regions may vary depending on the genomic integration location of interest, examples of such sequences include, but are not limited to: actin, ADA, albumin, α-globin, β-globin, CD2, CD3, CD5, CD7, CCR5, E1α, IL2RG, Ins1, Ins2, NCF1, p50, p65, PF4, PGC-γ, PTEN, TERT, UBC, and VWF, and the like.

In embodiments where the payload DNA, or a portion thereof, is to be integrated into the genome of a target cell in a process mediated by a gene editing system, e.g., as described below, the payload DNA, or integrating portion thereof, may be viewed a Donor DNA or Donor Template in such a system. Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein, and may in embodiments of the present invention be an payload DNA or component thereof. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double strand break occurs, the exogenous DNA can be inserted at the double strand break and thus become a permanent addition to the genome. These exogenous DNA molecules are referred to as donor templates in some embodiments. If the donor template contains a coding sequence for a gene-of-interest optionally together with relevant regulatory sequences such as promoters, enhancers, polyA sequences and/or splice acceptor sequences (also referred to herein as a “donor cassette”), the gene of interest can be expressed from the integrated copy in the genome resulting in permanent expression for the life of the cell. Moreover, the integrated copy of the donor DNA template can be transmitted to the daughter cells when the cell divides.

In the presence of sufficient concentrations of a donor DNA template that contains flanking DNA sequences with homology to the DNA sequence either side of the double strand break (referred to as homology arms), the donor DNA template can be integrated via the Homology Directed Repair (HDR) pathway. The homology arms act as substrates for homologous recombination between the donor template and the sequences either side of the double strand break. This can result in an error free insertion of the donor template in which the sequences either side of the double strand break are not altered from that in the un-modified genome. Alternatively, the donor template can be integrated by a mechanism that does not require homology arms, i.e. Non-homologous End Joining. In such instances, the donor template may or may not have homology arms. In some instances, the integration may be targeted to a particular locus, i.e. Homology Independent Targeted Integration (HITI), e.g. by the use of a nuclease to create an open break for integration and gRNA sequences that are complementary to the target region, as discussed further herein and known in the art.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles.

In some embodiments, an exogenous sequence, e.g., payload DNA, that is intended to be inserted into a genome is GOI or functional derivative thereof. The exogenous gene can include a nucleotide sequence encoding a GOI product, e.g., GOI protein, or functional derivative thereof. The functional derivative of a GOI can include a nucleic acid sequence encoding a functional derivative of a GOI protein that has a substantial activity of a wildtype GOI protein such as the wildtype human GOI protein, e.g., at least about 30%, about 40%, about 50%, about 60%, about 70%, about 80%, about 90%, about 95% or about 100% of the activity that the wildtype GOI protein exhibits. In some embodiments, the functional derivative of a GOI protein can have at least about 30%, about 40%, about 50%, about 60%, about 70%, about 80%, about 85%, about 90%, about 95%, about 96%, about 97%, about 98% or about 99% amino acid sequence identity to the GOI protein, e.g., the wildtype GOI protein. In some embodiments, one having ordinary skill in the art can use a number of methods known in the field to test the functionality or activity of a compound, e.g., peptide or protein. The functional derivative of the GOI protein can also include any fragment of the wildtype GOI protein or fragment of a modified GOI protein that has conservative modification on one or more of amino acid residues in the full length, wildtype GOI protein. Thus, in some embodiments, the functional derivative of a nucleic acid sequence of a GOI can have at least about 30%, about 40%, about 50%, about 60%, about 70%, about 80%, about 85%, about 90%, about 95%, about 96%, about 97%, about 98% or about 99% nucleic acid sequence identity to the GOI, e g. the wildtype GOI.

In some embodiments where the insertion of a GOI or functional derivative thereof is concerned, a cDNA of a GOI or functional derivative thereof can be inserted into a genome of a patient having defective GOI or its regulatory sequences. In such a case, a donor DNA or donor template can be an expression cassette or vector construct having the sequence encoding GOI or functional derivative thereof, e.g., cDNA sequence. In some embodiments, the expression vector contains a sequence encoding a modified GOI protein, which is described elsewhere in the disclosures, can be used.

In some embodiments, according to any of the donor templates described herein comprising a donor cassette, the donor cassette is flanked on one or both sides by a gRNA target site. For example, such a donor template may comprise a donor cassette with a gRNA target site 5′ of the donor cassette and/or a gRNA target site 3′ of the donor cassette. In some embodiments, the donor template comprises a donor cassette with a gRNA target site 5′ of the donor cassette. In some embodiments, the donor template comprises a donor cassette with a gRNA target site 3′ of the donor cassette. In some embodiments, the donor template comprises a donor cassette with a gRNA target site 5′ of the donor cassette and a gRNA target site 3′ of the donor cassette. In some embodiments, the donor template comprises a donor cassette with a gRNA target site 5′ of the donor cassette and a gRNA target site 3′ of the donor cassette, and the two gRNA target sites comprise the same sequence. In some embodiments, the donor template comprises at least one gRNA target site, and the at least one gRNA target site in the donor template comprises the same sequence as a gRNA target site in a target locus into which the donor cassette of the donor template is to be integrated. In some embodiments, the donor template comprises at least one gRNA target site, and the at least one gRNA target site in the donor template comprises the reverse complement of a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the reverse complement of a gRNA target site in a target locus into which the donor cassette of the donor template is to be integrated.

The term “auxiliary RNA” generally refers to a RNA molecule that is delivered to a cell, generally a cell of a subject delivered in vivo, to enhance some aspect of the delivery of a payload DNA which provides a function, e.g., to provide a therapeutic outcome for the subject. Accordingly, an auxiliary RNA generally provides a supportive function in the delivery and/or expression of the payload DNA within the LNP system. For example, supportive functions provided by inclusion of an auxiliary RNA in the LNP system (e.g., as co-administered or co-formulated) may include enhanced delivery of the payload DNA (e.g., as compared to the delivery resulting from a corresponding system that does not include the auxiliary RNA), enhanced expression of a coding sequence present in the payload DNA (e.g., as compared to the level of expression resulting from a corresponding system that does not include the auxiliary RNA), enhanced incorporation of the payload DNA into a targeted locus of the genome of the target cell (e.g., as compared to the level of incorporation resulting from a corresponding system that does not include the auxiliary RNA), enhanced immune evasion of the payload DNA (i.e., reduced immune response in the subject to the payload DNA) (e.g., as compared to the level of immune response resulting from delivery of a corresponding system that does not include the auxiliary RNA), or some combination of multiple enhancements.

Auxiliary RNAs, useful in the systems and compositions of the present disclosure, will vary. Useful auxiliary RNAs may or may not include a coding sequence. In some instances, an auxiliary RNA may be, or may include as a portion of the overall auxiliary RNA, a non-coding RNA (ncRNA), including synthetic ncRNA (e.g., an RNA containing artificial sequence that does not encode a protein) or a type of naturally occurring ncRNA (e.g., rRNA, tRNA, microRNAs, siRNAs, piRNAs, snoRNAs, snRNAs, exRNAs, scaRNAs, long ncRNAs) or sequence thereof. In some instances, an auxiliary RNA may be, or may include as a portion of the overall auxiliary RNA, a coding RNA, e.g., an mRNA that encodes a polypeptide. In some instances, an auxiliary RNA may entirely exclude (i.e., not consist of) one or more particular types of RNA, such as one or more type of ncRNA or mRNA. In some instances, an auxiliary RNA may consist entirely of a single species of RNA, e.g., entirely mRNA. In some instances, an auxiliary RNA may be a hybrid of two or more different forms or types of RNA. The amount of different RNA sequences that make up a hybrid auxiliary RNA will vary. For example, in some instances, an auxiliary RNA may be entirely made of up ncRNA or sequence that does not encode a protein. In some instances, an auxiliary RNA includes one or more portions that do not encode a polypeptide, including e.g., wherein at least 10%, 20%, 30%, 40%, 50%, 60%, 70%, 80%, 90%, or more of the auxiliary RNA does not encode a polypeptide. Where an auxiliary RNA includes synthetic RNA sequence, the amount of synthetic sequence may vary including where the entire auxiliary RNA consists of synthetic sequence or less than the 100% of the RNA is synthetic, including e.g., wherein at least 10%, 20%, 30%, 40%, 50%, 60%, 70%, 80%, 90%, or more of the auxiliary RNA comprises synthetic RNA sequence. Auxiliary RNAs may consist entirely of naturally occurring RNA ribonucleosides or may include one or more chemically modified or synthetic nucleosides, a synthetic nucleoside cap, or a combination thereof. Examples of chemically modified nucleotides include, for example, N1-methylpseudouridine, 2-thiouridine (s2U), 5-methylcytidine (m5C), N6-methyladenosine (m6A), 2′-O-methyluridine (Um), 2′-O-methylcytidine (Cm), 2′-O-methyladenosine (Am), and 2′-O-methylguanosine (Gm). The RNA may comprise a 5′ cap or analog thereof, e.g. an m7GpppG cap, anti-reverse cap analog (ARCA), a two-headed cap, an S cap, a 2S cap, and the like. The RNA may comprise a tail modification, e.g. a ribose modified adenosine, 8-azaadenosine, cordycepin, and the like.

The length and composition of auxiliary RNAs will vary widely. For example, in some instances, auxiliary RNAs may be at least 30 nt in length, including e.g., at least 40 nt, at least 50 nt, at least 60 nt, at least 70 nt, at least 80 nt, at least 90 nt, at least 100 nt, at least 150 nt, at least 200 nt, at least 300 nt, at least 400 nt, at least 500 nt, at least 600 nt, at least 700 nt, at least 800 nt, at least 900 nt, at least 1000 nt, or more in length. In some instances, auxiliary RNAs will be less than about 10 kb in length, including e.g., less than about 9 kb, about 8 kb, about 7 kb, about 6 kb, about 5 kb, or less. As described herein, an auxiliary RNA may or may not encode a polypeptide and may or may not include any, all, or some elements of an mRNA (e.g., a 5′ cap, a 5′ UTR, a coding sequence, a 3′ UTR, and a polyA tail), whether or not such RNA actually encodes a polypeptide. Where an auxiliary RNA encodes a polypeptide, the polypeptide may be a non-therapeutic polypeptide, such as e.g., a reporter or a DBD-containing protein that enhances transport of a payload DNA that includes a sequence to which the DBD-containing protein binds.

As described and demonstrated herein, systems, compositions, and methods of the present disclosure employing co-administration of payload DNA LNPs and auxiliary RNA LNPs or co-formulated LNPs containing payload DNA with auxiliary RNA results in enhanced effects as compared to administration of payload DNA LNPs alone. The ratio of auxiliary RNA to payload DNA will vary and may, e.g., be at least 1:1, including e.g., where the auxiliary RNA to payload DNA ratio is greater than 1:1, including but not limited to e.g., at least 1.5:1, at least 2:1, at least 3:1, at least 4:1, at least 5:1, or greater. Such ratios may be determined in various ways including e.g., where the ratio is a weight-to-weight ratio (w/w ratio), e.g., a w/w ratio of auxiliary RNA to payload DNA is 1:1 or greater.

Where the auxiliary RNA and payload DNA are present in such ratios, the individual auxiliary RNA and DNA may further have any of the characteristics, or combination of characteristics, described herein, including e.g., where the payload DNA is plasmid DNA (pDNA), nanoplasmid DNA (npDNA), doggy-bone DNA (dbDNA), mini-circle DNA (mcDNA), single-stranded circular DNA (circDNA), closed ended DNA (ceDNA), or 3D DNA and at least 100 nt in length, and the auxiliary RNA is at least 30 nt in length; and the w/w ratio of auxiliary RNA to payload DNA is 1:1 or greater.

The payload DNA and auxiliary RNA, or their expression products, may or may not be configured to physically interact. For example, in some instances, the auxiliary RNA may encode for an irrelevant non-therapeutic polypeptide (e.g., a reporter) that does not bind or otherwise physically interact with the payload DNA or an encoded product thereof, e.g., a therapeutic polypeptide. In some instances, the auxiliary RNA may encode for a non-therapeutic polypeptide that includes a DBD that binds to a sequence present in the expression product of the payload DNA such that the product of the auxiliary RNA and the payload DNA physically interact.

In some instances, nucleic acids of the present disclosure may be configured for nuclear import, either of the nucleic acids themselves or of the expression products of the nucleic acids or both. For example, in some instances, a payload DNA may include one or more sequences that promotes the translocation of the payload DNA into the nucleus of a eukaryotic cell. In some instances, such sequences may be referred to as a DNA nuclear targeting sequence (DTS). Sequences useful as a DTS in a payload DNA include endogenous and heterologous sequences that, when bound by a protein, promote the translocation of the payload DNA into the nucleus. For example, as described in more detail elsewhere herein, useful DTSs include sequences bound by a DNA binding domain (DBD) of a protein that is targeted to the nucleus, e.g., due to the presence of an endogenous or heterologous nuclear localization sequence/signal present in the protein. Accordingly, auxiliary RNAs of the present disclosure may include, or may be engineered to include, sequence encoding one or more polypeptides that influence nuclear transport, such as a nuclear localization sequences/signals and a nuclear export signals. In some instances, an endogenous protein of the cell may influence nuclear transport of a payload DNA containing a DTS, such that the endogenous protein facilitates nuclear transport by binding to a sequence of the payload DNA via a DBD present in the protein.

A sequence encoding a nuclear localization sequence or nuclear localization signal (NLS) peptide may be included in one or more of the nucleic acids of the disclosure. In some instances, a sequence encoding an NLS may be included, or attached to, an auxiliary RNA. For example, an auxiliary RNA provided as an mRNA may include a sequence encoding an NLS such that, upon translation of the mRNA in the cytoplasm, the NLS promotes translocation of the encoded product into the nucleus of the cell. In some instances, a sequence encoding an NLS may be included, or attached to, a payload DNA. In some instances, an employed NLS may be native, or endogenous, to a coding sequence included in one or more nucleic acids of the system, including a coding sequence containing the NLS, e.g., a payload DNA, an auxiliary RNA, or both. In some instances, an employed NLS may be non-native, or exogenous or heterologous, to a coding sequence included in one or more nucleic acids of the system, including a coding sequence containing the NLS, e.g., a payload DNA, an auxiliary RNA, or both. If engineered into a desired coding sequence, the NLS may be engineered to be anywhere within the coding sequence.

Any useful and appropriate NLS, or sequence encoding an NLS, or fragment thereof, may be employed. Useful NLSs include classical NLSs, nonclassical NLSs and other types of NLSs, including but not limited to monopartite NLSs (such as those having a K (K/R) X (K/R) motif, where X can by any residue), bipartite NLSs (such as those having a R/K(X)₁₀₋₁₂KRXK motif, where X can by any residue), proline-tyrosine NLSs (PY-NLS) (such as those having a R/K/H(X)₂₋₅PY motif, where X can by any residue), and the like. NLSs and sequences encoding NLSs may be derived from various sources, including those proteins that bind transport receptors (e.g., importin alpha proteins and importin beta proteins) including but not limited to SV40 large T-antigen, cMyc, influenza A virus proteins, VACM-1/CUL5, CXCR4, VP1, 53BP1, ING4, IER5, ERK5, Hrp1, UL79, EWS, PTHrP, Pho4, rpL23a, PABPN1, STAT1, FGF2, MSX1, NLS-RARα, and the like. Non-limiting examples of useful NLSs include: PKKKRKV (SEQ ID NO: 01), PKLKRQ (SEQ ID NO:02), RPRK (SEQ ID NO:03), RRARRPRG (SEQ ID NO:04), GKRKLITSEEERSPAKRGRKS (SEQ ID NO:05), KGKKGRTQKEKKAARARSKGKN (SEQ ID NO: 06), RKRCAAGVGGGPAGCPAPGSTPLKKPRR (SEQ ID NO:07), RKPVTAQERQREREEKRRRRQERAKEREKRRQERER (SEQ ID NO:08), RSGGNHRRNGRGGRGGYNRRNNGYHPY (SEQ ID NO:09), TLLLRETMNNLGVSDHAVLSRKTPQPY (SEQ ID NO:10), PGKMDKGEHRQERRDRPY (SEQ ID NO: 11), GKKKKGKPGKRREQRKKKRRT (SEQ ID NO:12), SANKVTKNKSNSSPYLNKRKGKPGPDS (SEQ ID NO:13), VHSHKKKKIPTSPTFTTPKTLTLRRQPKYPRKSAPRRNKLDHY (SEQ ID NO:14), RKHKTNRKPR (SEQ ID NO:15), NRRAKAKR (SEQ ID NO:16), RNKKKK (SEQ ID NO:17), RKVIK (SEQ ID NO:18), and the like. Useful NLSs also include those listed in Table 1, those described in Lu et al. (2021) Cell Commun Signal.19(1):60; and the like.

TABLE 1
Exemplary NLS sequences

| NLS origin | Protein sequence |
|---|---|
| SV40 Large T antigen SMALL | PKKKRKVED (SEQ ID NO: 19) |
| SV40 Large T antigen | KKKSSSDDEATADSQHSTPPKKKRKVEDPKDFPSE |
| | LLS (SEQ ID NO: 20) |
| Optimized SV40 Large T antigen | PSSDDEATADSQHAAPPKKKRKVEDPKDFPSELLS |
| | (SEQ ID NO: 21) |
| Heterogeneous nuclear RNP A1.1 | GNQSSNFGPMKGGNFGGRSSGPYGGGGQYFAKP |
| M9 domain | RNQGGYGG (SEQ ID NO: 22) |
| Heterogeneous nuclear RNP A1.2 | FGNYNNQSSNFGPMKGGNFGGRSSGPYGGGGQY |
| M9 domain | FAKPRNQGGYGG (SEQ ID NO: 23) |
| Heterogeneous nuclear RNP A1.3 | FGNYNNQSSNFGPMKGGNFGGRSSGPYGGGGQY |
| M9 domain | FAKPRNQGGYGGSSSSSSYGSGRRF (SEQ ID |
| | NO: 24) |
| HIV viral protein 1 (VP1) | DTWTGVEALIRILQQLLFIHFRIGCRHSRIGIIQQRRT |
| | RNGA (SEQ ID NO: 25) |
| HIV viral protein 2 (VP2) | GDTWAGVEAIIRILQQLLFIHFRIGCRHSRIGVTRQR |
| | RARNGASRS (SEQ ID NO: 26) |
| HIV viral protein 3 (VP3) | EQAPEDQGPQREPHNEWTLELLEELKNEAVRHFP |
| | RIWLHGLGQHIYETYGDTWAGVEAIIRILQQLLFIHFR |
| | IGCRHSRIGVTRQRRARNGASRS (SEQ ID NO: 27) |
| Influenza virus A nuclear protein | ASQGTKRSYEQMETDGERQ (SEQ ID NO: 28) |
| (INF-A) | |
| Cellular myc transcription factor | PAAKRVKLD (SEQ ID NO: 29) |
| (c-myc) | |
| c-myc fused to INF-A (cMyc) | PAAKRVKLDASQGTKRSYEQMETDGERQ (SEQ ID |
| | NO: 30) |
| Biparticle nucleus localization signal | KRTADGSEFESPKKKRKV (SEQ ID NO: 31) |

Where employed, an NLS encoding sequence may be included essentially anywhere within a nucleic acid and correspondingly the NLS may be essentially anywhere within an encoded polypeptide. In some instances, the NLS is engineered to be at the N terminus. In some instances, the NLS is engineered to be at the C terminus. In some instances, the NLS is engineered to be at both the N terminus and the C terminus. When engineered to reside at a terminus, the NLS may be within 0-20 amino acids of the terminus, in some instances, 0-10 amino acids of the terminus, in certain instances 0-5 amino acids of the terminus, in some such cases, at the terminus. In some instances, the NLS may be flanked by one or more amino acids, e.g., 2, 3, 4, 5, 6, 7, 8, 9, 10, 11, 12, 13, 14, 15, 16, 17, 18, 19, or 20 amino acids, e.g., between the NLS and the next protein domain. In some instances, the NLS and the next protein domain may be adjacent, e.g., with no intervening amino acids. An NLS may be engineered to be at a site that is distinct from other the protein domains, such as a DBD. In some instances, an encoded polypeptide will include one NLS, in other instances multiple NLSs. In some instances in which multiple NLSs are employed, the same NLS is employed multiple times. In other instances in which multiple NLSs are employed, different NLSs are used. Multiple NLSs may or may not be separated by a linker.

In some embodiments, a polypeptide encoded by one or more nucleic acids of the disclosure may include a nuclear export signal (NES). In some embodiments, the NES is NMD3 ribosome export adaptor. In some such embodiments, the NES comprises a sequence having 85% identity or more to EHVNKMNSDRVPDVVLIKKSYDRTKRQRRRNWKLKELA (SEQ ID NO: 32).

As described herein, LNP systems of the present disclosure employ a combination of an auxiliary RNA and payload DNA that results in enhanced function of the payload DNA, e.g., increasing expression of the expression product of the payload DNA, by increasing delivery of the payload DNA, by decreasing the host immune reaction to the payload DNA containing-LNP, or a combination thereof. In some instances, the auxiliary RNA and payload DNA may be engineered to further enhance beneficial effects, e.g., by adding additional functions to the auxiliary RNA and, in some instance, modifying the payload DNA such that it is configured to interact with such added functions. The further enhanced beneficial effects due to added additional functions may include, e.g., further enhanced delivery of the payload DNA, further enhanced expression of the expression product of the payload DNA, etc. As summarized, engineering of additional functions into the auxiliary RNA and payload DNA may include the incorporation of elements into the payload DNA and auxiliary RNA that cause the payload DNA and auxiliary RNA to interact. For example, in some instances, an auxiliary RNA may be engineered to include sequence encoding a polypeptide that includes a DBD and the payload DNA may be engineered to include a sequence to which the encoded DBD binds, e.g., a DTS.

In some instances where the payload DNA and a polypeptide encoded by the auxiliary RNA are engineered to interact, the product of the auxiliary RNA may include one or more nuclear transport elements, such as an NLS, a NES, combinations thereof, and the like. In some instances, a nuclear transport element may be a heterologous or endogenous component of a DBD-containing polypeptide. For example, a system may be employed that includes, at least, (1) an auxiliary RNA encoding a DBD-containing polypeptide that includes a nuclear transport element and (2) a payload DNA that includes a DTS to which the DBD of the DBD-containing polypeptide binds. Thus, upon introduction of the auxiliary RNA and the payload DNA into a cell, the DBD-containing polypeptide is expressed and binds the DTS of the payload DNA thereby transporting the payload DNA into the nucleus of the cell.

A DTS refers to a nucleotide sequence that mediates the translocation of a polynucleotide that comprises it into the nucleus of a cell. Without wishing to be bound by theory, it is believed that DTSs leverage the movement of nuclear-acting DNA binding proteins as they move from the cytoplasm into the nucleus. These nuclear-acting DNA binding proteins act like nuclear targeting factors for DNA, binding to sequences on the DNA and dragging the DNA into the nucleus as the DNA binding protein translocates into the nucleus. Accordingly, the nuclear-acting DNA binding proteins that are leveraged are referred to herein as DBD-containing polypeptides or nuclear targeting factors (NTFs), and the DNA sequences to which they bind are referred to herein as nuclear targeting factor binding sites (NTFBSs, or more simply, TFBSs).

A given DTS may include 2 or more different TFBSs (i.e., TFBSs that differ from each other by nucleotide sequence and are therefore distinct), such as 2, 3, 4, 5, 6, 7, 8, 9 or 10 different TFBSs. A given DTS may include 1 or more copies of the same TFBS, such as 2 or more, 3 or more, 4 or more, 5 or more, 6 or more, 7 or more, 8 or more, 9 or more, copies of the same TFBS, where in some instances the number of identical TFBS copies does not exceed 10.

The length of a given DTS may vary depending on the number of TFBSs comprised by it, the lengths of the TFBS sequence to which each nuclear targeting factor binds, and the number nucleotides between TFBSs (the spacer sequence). Where two or more TFBSs (either the same or different) are present in a given DTS, the distance between any two TFBSs may vary as desired, ranging in some instances from 5 to 100 bp, such as 10 to 75 bp, including 15 to 50 bp. For example, the TFBSs may be separated from one another by 0, 1, 2, 3, 4, 5, 6, 7, 8, 9, 10, 11, 12, 13, 14, 15, 16, 17, 18, 19, or 20 or more nts, for example, 0-5 nts, 6-10 nts, 11-15 nts, 16-20 nts, 21-25 nts, 26-30 nts, 31-35 nts, 36-40 nts, in some instances 40-50 nts. In some instances, the length of a given DTS ranges from 10 to 500 nt, such as 100 to 300 nt and including 150 to 200 nt.

The DTS may be incorporated in the payload DNA in any of a variety of positions. For example, where the payload DNA includes a coding sequence the DTS may be placed 5′ or 3′ of the coding sequence. Where the payload DNA includes an expression cassette the DTS may be placed 5′ of the promoter, 3′ of the promoter and 5′ of the expression cassette, within an intron of the expression cassette, 3′ of the expression cassette. In some instances, the DTS is placed 5′ of the promoter. In some embodiments, the DTS is placed 3′ of the expression cassette.

In some embodiments, the DTS comprises a TFBS that is bound by a NTF that is active in the target cell. For example, the nuclear targeting factor may be constitutively expressed in the target cell. As another example, the NTF may be typically latent in the cytoplasm of the target cell but become active when the cell is contacted by the payload DNA containing the TFBS, for example as part of a cellular response to the payload DNA containing the TFBS or the formulation comprising the payload DNA containing the TFBS, for example as part of an inflammatory response, including e.g., transcription factors that are responsive to TLR9, cGAS/STING, AIM2, IFI16, or DDX41 activation, e.g., NF-κB, IRF3, IRF7, and others as known in the art.

In some instances, as described herein, an endogenous NTF may be employed, e.g., to facilitate enhanced transport of a payload DNA into the nucleus of a cell. In some instances, an additional copy of the endogenous NTF may be employed, e.g., as an encoded product of an auxiliary RNA, and the expression product of the additional copy of the NTF-coding sequence may further facilitate translocation of the payload DNA into the nucleus of the cell.

Table 2 provides nonlimiting examples of TFBSs that may be utilized in DTSs and the TNFs that could be leveraged to achieve nuclear transport of payload DNA without needing to provide exogenous TNFs or sequence encoding exogenous NTFs.

TABLE 2
NTFBSs and the corresponding NTFs leveraged for nuclear translocation

| NTF DNA binding protein | TFBS |
|---|---|
| ATF3 | TGACGTCA (SEQ ID NO: 33) |
| CEBPA v1 | ATTGCGCAAT (SEQ ID NO: 34) |
| CEBPA v2 | ATTGTGCAAT (SEQ ID NO: 35) |
| CEBPA v3 | ATTGCACAAT (SEQ ID NO: 36) |
| CEBPA v4 | ATTACAAAAT (SEQ ID NO: 37) |
| CEBPA v5 | TGTTTGTTAAGGC (SEQ ID NO: 38) |
| CEBPA v6 | TGGTATGATTTTGTAATGGGGTAGGA (SEQ |
| | ID NO: 39) |
| CREB1 v1 | TGACGTCA (SEQ ID NO: 40) |
| CREB1 v2 | CTGACGTCAG (SEQ ID NO: 41) |
| CREB1 v3 | GCACGTCA (SEQ ID NO: 42) |
| CREB3L3 v1 | CCACGTTG (SEQ ID NO: 43) |
| CREB3L3 v2 | CCACGTAG (SEQ ID NO: 44) |
| CREB3L3 v3 | CCACGCTG (SEQ ID NO: 45) |
| CREB3L3 v4 | CCATGAACTTTG (SEQ ID NO: 46) |
| CREB3L3 v5 | CAAACGTGGTTT (SEQ ID NO: 47) |
| CREB3L3 v6 | TCCACGTGGTATT (SEQ ID NO: 48) |
| CREB3L3 v7 | TACACGTAATC (SEQ ID NO: 49) |
| CREB3L3 v8 | CACACGTGATC (SEQ ID NO: 50) |
| E2F1 v1 | GGCGCCAA (SEQ ID NO: 51) |
| E2F1 v2 | AAAAATGGCGCCAAAATG (SEQ ID NO: 52) |
| EBF1 (COE1) | TCCCTAGGGA (SEQ ID NO: 53) |
| EGR3 (PILOT) v1 | CGCCCACGCA (SEQ ID NO: 54) |
| EGR3 (PILOT) v2 | TACGCCCACGCATT (SEQ ID NO: 55) |
| ELF5 v1 (ESE-2) | CCGGAAGT (SEQ ID NO: 56) |
| ELF5 v2 | AACCCGGAAGTG (SEQ ID NO: 57) |
| ELK1 v1 | CCGGAAG (SEQ ID NO: 58) |
| ELK1 v2 | CACTTCCGCCGGAAGTG (SEQ ID NO: 59) |
| ETS1 v1 | ACAGGAAGT (SEQ ID NO: 60) |
| ETS1 v2 | ACCGGAAGTACTTCCGGT (SEQ ID NO: 61) |
| ETV4 (E1A-F, PEA3) | ACCGGAAGT (SEQ ID NO: 62) |
| FOXA1 (HNF-3α) v1 | TGTTTACTTT (SEQ ID NO: 63) |
| FOXA1 (HNF-3α) v2 | TTGTTTACTT (SEQ ID NO: 64) |
| FOXA2 (HNF-3β) | TGTTTGTTT (SEQ ID NO: 65) |
| FOXP3 | GTAAACA (SEQ ID NO: 66) |
| GATA6 | AGATAAGA (SEQ ID NO: 67) |
| GTF21 (TFII-I) | CCATT (SEQ ID NO: 68) |
| HNF1A (HNF-1α, LF-B1) v1 | GTTAATGATTAAC (SEQ ID NO: 69) |
| HNF1A (HNF-1α, LF-B1) v2 | GTTAATAATCTAC (SEQ ID NO: 70) |
| HNF1A (HNF-1α, LF-B1) v3 | GTTACTTATTCTC (SEQ ID NO: 71) |
| HNF1A (HNF-1α, LF-B1) v4 | GGTTAATAATTAAC (SEQ ID NO: 72) |
| HNF1A (HNF-1α, LF-B1) v5 | AATTATTTATTACCA (SEQ ID NO: 73) |
| HNF1A (HNF-1α, LF-B1) v6 | AGTATGGTTAATGATCTACAG (SEQ ID NO: 74) |
| HNF1A (HNF-1α, LF-B1) v7 | TGGTTAATATTCACCAGC (SEQ ID NO: 75) |
| HNF1A (HNF-1α, LF-B1) v8 | GTTTATCAGTGACTAGTCATTGAT (SEQ ID |
| | NO: 76) |
| HNF1A (HNF-1α, LF-B1) v9 | TGATAGCCAACTGCAGCTAATAATAAACCA |
| | (SEQ ID NO: 77) |
| HNF1A (HNF-1α, LF-B1) v10 | ATATTTTAGAGAAGAATTAACCTTT (SEQ ID |
| | NO: 78) |
| HNF4A (HNF-4α, NR2A1) v1 | AGTCCAAAGTTCA (SEQ ID NO: 79) |
| HNF4A (HNF-4α, NR2A1) v2 | TCGAGCGCTGGGCAAAGGTCACCTGC (SEQ |
| | ID NO: 80) |
| HNF4A (HNF-4α, NR2A1) v3 | TCGAAGGGCAGGGGTCAAGGGTTCAGT |
| | (SEQ ID NO: 81) |
| HNF4A (HNF-4α, NR2A1) v4 | TCGAGCGCAGGTCAAAGGTCACCTGC (SEQ |
| | ID NO: 82) |
| HNF4A (HNF-4α, NR2A1) v5 | TCGAGCGCAGGTCAAAAGGTCACCTGC |
| | (SEQ ID NO: 83) |
| HNF4A (HNF-4α, NR2A1) v6 | AGGTTAAAGGTCT (SEQ ID NO: 84) |
| HNF4A (HNF-4α, NR2A1) v7 | AGGTCAAAGTCCA (SEQ ID NO: 85) |
| HNF4A (HNF-4α, NR2A1) v8 | TGGGTCCAGAGGGCAAAA (SEQ ID NO: 86) |
| HNF4A (HNF-4α, NR2A1) v9 | CGCCCCAGCACACATGATCAGA (SEQ ID |
| | NO: 87) |
| HNF4A (HNF-4α, NR2A1) v10 | AACACGGGAGGTCAAAGATTGCGCCC (SEQ |
| | ID NO: 88) |
| IKZF1 (IK1, IKAROS, Lyf-1, | TTGGGAAT (SEQ ID NO: 89) |
| ZNFN1A1) | |
| IRF1 | GAAAGTGAAAGT (SEQ ID NO: 90) |
| JUN | TGAGTCA (SEQ ID NO: 91) |
| LEF1 (TCF-1α) | AGATCAAAG (SEQ ID NO: 92) |
| LRRFIP1 (GCF2, TRIP) | AGCCCCCGGCG (SEQ ID NO: 93) |
| MAZ (Zif87, ZNF801, Pur-1, SAF) | GGGGAGGG (SEQ ID NO: 94) |
| MYB | CAGTTG (SEQ ID NO: 95) |
| NFATC2 (NFATp, NFAT1) | TTTCCATGGAAA (SEQ ID NO: 96) |
| NF-IC | TTGGCTATATGCCAA (SEQ ID NO: 97) |
| NFYA (CP1A, CBF-B) | CCAAT (SEQ ID NO: 98) |
| ONECUT-1 (HNF6, HNF-6α, OC-1) v1 | TATTGATT (SEQ ID NO: 99) |
| ONECUT-1 (HNF6, HNF-6α, OC-1) v2 | TCCATTGATTTAG (SEQ ID NO: 100) |
| ONECUT-1 (HNF6, HNF-6α, OC-1) v3 | AAAAAATCAATAAT (SEQ ID NO: 101) |
| ONECUT-1 (HNF6, HNF-6α, OC-1) v4 | GAAAAAAAAATCAATATCGGGCCT (SEQ ID |
| | NO: 102) |
| ONECUT-1 (HNF6, HNF-6α, OC-1) v5 | GTCTGCTAAGTCAATAATCAGAAT (SEQ ID |
| | NO: 103) |
| PAX5 | GTCATGCGTGAC (SEQ ID NO: 104) |
| PLAG1 | CCCCCTTGGGCCCC (SEQ ID NO: 105) |
| POU2F1 (Oct-1, OTF-1) v1 | TATGCAAAT (SEQ ID NO: 106) |
| POU2F1 (Oct-1, OTF-1) v2 | AATATGCAAATTAG (SEQ ID NO: 107) |
| POU2F1 (Oct-1, OTF-1) v3 | TTATGCATATGCATAA (SEQ ID NO: 108) |
| POU2F1 (Oct-1, OTF-1) v4 | ATATGATTATGCAAATTTATAGA (SEQ ID |
| | NO: 109) |
| PPARα (PPARA, NR1C1) v1 | AGGTCATCAGGTCA (SEQ ID NO: 110) |
| PPARα (PPARA, NR1C1) v2 | AGGTCAAAGGTCA (SEQ ID NO: 111) |
| PPARα (PPARA, NR1C1) v3 | AGGTCAATGACCT (SEQ ID NO: 112) |
| PPARα (PPARA, NR1C1) v4 | GTGTCAAAGGTCA (SEQ ID NO: 113) |
| PPARα (PPARA, NR1C1) v5 | AACTAGGTCAAAGGTCA (SEQ ID NO: 114) |
| PPARα (PPARA, NR1C1) v6 | CAAAACTAGGTCAAAGGTCA (SEQ ID NO: 115) |
| PPARα (PPARA, NR1C1) v7 | AACTAGGTCAAAGGTCAAAG (SEQ ID NO: 116) |
| SMAD1 | GTCTAGAC (SEQ ID NO: 117) |
| SP1v1 | GGGGCGGGG (SEQ ID NO: 118) |
| SP1v2 | GCCACGCCCCC (SEQ ID NO: 119) |
| SPI1 (PU.1) | AAAAGAGGAAGTG (SEQ ID NO: 120) |
| SRY (TDF) | AAACAATAACATTGTTT (SEQ ID NO: 121) |
| STAT1 | TTCCCGGAA (SEQ ID NO: 122) |
| TBP | TATAAAA (SEQ ID NO: 123) |
| TCF7L2 (TCF-4) | ACTTCAAAGG (SEQ ID NO: 124) |
| TFAP2A (AP-2a) | GCCTGAGGC (SEQ ID NO: 125) |
| TP53 (p53) | GGACATGCCCGGGCATGTCC (SEQ ID |
| | NO: 126) |
| USF2 | GTCACGTGAC (SEQ ID NO: 127) |
| WT1 | CGCCCCCGC (SEQ ID NO: 128) |
| XBP1 | GATGACGTCATC (SEQ ID NO: 129) |
| YY1 | GCCGCCATTTT (SEQ ID NO: 130) |
| ZNF423 (OAZ, Zfp423) | GCACCCAAGGGTGC (SEQ ID NO: 131) |

In some instances, the NTF may be provided to the cell, e.g., as a protein or as an mRNA encoding a protein. For example, a sequence encoding the NTF may be provided as a coding sequence present on an auxiliary RNA provided to the cell. Such NTF coding sequence may encode an NTF that is heterologous to the cell or provide a copy of a coding sequence for an NTF that is endogenous to the cell. In some instance, provision of an additional copy of an endogenous coding sequence allows for expression of the NTF that is increased about basal level endogenous NTF expression, e.g., through the use of a strong promoter operably linked to the NTF-coding sequence or simply through the provision of an extra copy of the coding sequence. Where a heterologous NTF is employed the genome of the cell does not include a sequence encoding the NTF and thus the coding sequence employed is heterologous to the cell.

In some embodiments, a DTS comprises a TFBS that is bound by a NTF that is exogenously provided to the cell, e.g., as a component encoded on the auxiliary RNA. As will be appreciated by one of ordinary skill in the art, any protein that binds to DNA and that traffics to the nucleus when delivered to the cytoplasm, or translated from RNA delivered to the cytoplasm, can be employed to mediate nuclear translocation. Thus, for example, any of the naturally occurring proteins described in Table 2 may be exogenously provided. As another example, a protein that is not native to the cell, i.e., that is heterologous to the cell, may be provided. For example, a prokaryotic protein, a synthetic protein, or a heterologous eukaryotic protein may be provided to a eukaryotic cell, including e.g., a bacterial protein to an animal cell, a yeast protein to an animal cell, an non-mammalian protein to a mammalian cell, a non-human protein mammalian protein to a human cell, a synthetic protein to a mammalian cell, such as a human cell, etc. Such a protein may be naturally occurring or engineered/synthetic.

Examples include any of the proteins listed in Table 3. Exemplary proteins of each class and the DNA sequences to which they bind are well known in the art and include those described in greater detail below.

TABLE 3
Classes of NTFs that may be delivered, e.g., on an auxiliary
RNA, with payload DNA for DNA nuclear transport

| Protein/DNA binding system | Example NTF protein |
|---|---|
| Bacterial regulatory proteins | Tetracycline repressor (TetR), |
| | Purine synthesis repressor (PurR); |
| | Lac repressor (LacR) |
| Yeast regulatory proteins | Gal4; GCN4 |
| Bacteriophage regulatory proteins | Arc; Mnt; Bac434 |
| Mammalian regulatory proteins | YY1; HEY1; MyoD; GR; ER; PR |
| Zing finger proteins (ZFP) | ZFP-CCR5 |
| Transcription Activator-Like Effectors (TALE) | TALE-CCR5 |
| Inactivated nucleases | Inactivated I-Scel |
| Inactivated Site-Specific Recombinases | Cre; Piggybac |
| Transcription factor DNA-binding domain | STAT1; ESR1; Gal4 |
| Inactivated RNA-guided endonuclease | dCas9; HypaCas9; miniCas9; Cas12a |
| Single strand DNA binding protein | hSSB1; POT1 |

In some embodiments, the TFBS is a binding sequence for a Tet Repressor (TetR) protein, for example, a TetO sequence such as YCTATCANTGATAGA (SEQ ID NO:132), for example, TCCCTATCAGTGATAGAGA (SEQ ID NO:133) or TCGAGTTTACTCCCTATCAGTGATAGAGAACG (SEQ ID NO:134). In some embodiments, the DTS comprises 2, 3, 4, 5, 6, 7, 8, 9, or 10 copies of the TetR TFBS. In certain embodiments, the DTS comprises 7 copies of the TetR TFBS. In certain embodiments, the DTS comprises 10 or more copies of the TetR TFBS. In some embodiments, the DTS comprises a sequence having 80% identity or more to a sequence listed in Table 4, for example, 85% or 90% identity or more, in some cases 95% identity or more, e.g. 96%, 97%, 98%, or 99% identity to a sequence in Table 4. In some instances, the DTS binding sequence is identical to a sequence in Table 4. In some instances, the DTS consists essential of a sequence in Table 4. In certain embodiments, the DTS comprises a tetracycline response element (TRE), as known in the art. In certain embodiments, the DTS consists essentially of a TRE.

TABLE 4
Examples of DTSs that comprise a TetR binding sequence (e.g., TetO) in
varying numbers and sequences.

| DTS | Sequence (5′ to 3′) |
|---|---|
| TetO Motif | YCTATCANTGATAGA (SEQ ID NO: 135) |
| TetO-1 (Core) | TCCCTATCAGTGATAGAGA (SEQ ID NO: 136) |
| TetO-2 | CTCGAGTTTACTCCCTATCAGTGATAGAGAACGTATGTCGAGTTTACTCCCTA |
| | TCAGTGATAGAGAACGATGTCGAGTTTAC (SEQ ID NO: 137) |
| TetO-3 | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTAC (SEQ ID NO: 138) |
| TetO-4 | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTACTCCCTATCAGTGATAGAGAACGTATGTCGAGTTTAC (SEQ |
| | ID NO: 139) |
| TetO-5 | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTACTCCCTATCAGTGATAGAGAACGTATGTCGAGTTTACTCCC |
| | TATCAGTGATAGAGAACGTATGTCGAGTTTA (SEQ ID NO: 140) |
| TetO-6 | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTACTCCCTATCAGTGATAGAGAACGTATGTCGAGTTTACTCCC |
| | TATCAGTGATAGAGAACGTATGTCGAGTTTATCCCTATCAGTGATAGAGAAC |
| | GTATGTCGAGTTTAC (SEQ ID NO: 141) |
| TetO-7 | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTACTCCCTATCAGTGATAGAGAACGTATGTCGAGTTTACTCCC |
| | TATCAGTGATAGAGAACGTATGTCGAGTTTATCCCTATCAGTGATAGAGAAC |
| | GTATGTCGAGTTTACTCCCTATCAGTGATAGAGAACGTATGTCGAGGTAGGC |
| | GTGTACGGTGGGAGGCCTATATAAGCAGAGCTCGTTTAGTGAACCGTCAGAT |
| | CGCCTGGAGAATTCA (SEQ ID NO: 142) |
| TetO-7-m | CTCGAGTTTACTCCCTATCAGTGATAGAGAACGTATGTCGAGTTTACTCCCTA |
| | TCAGTGATAGAGAACGATGTCGAGTTTACTCCCTATCAGTGATAGAGAACGT |
| | ATGTCGAGTTTACTCCCTATCAGTGATAGAGAACGTATGTCGAGTTTACTCCC |
| | TATCAGTGATAGAGAACGTATGTCGAGTTTATCCCTATCAGTGATAGAGAAC |
| | GTATGTCGAGTTTACTCCCTATCAGTGATAGAGAACGTATGTCGAGGTA |
| | (SEQ ID NO: 143) |

In some embodiments, the TFBS is a binding sequence for the DNA binding domain of a gene editing system, e.g., as described herein or as known in the art, e.g. the guide RNA of a Cas nuclease, the zinc finger domain of a zinc finger nuclease, the TALE DNA binding domain of a TALEN.

In some embodiments, the TFBS is a binding sequence for a zinc-finger containing protein (“ZF protein”). As will be appreciated by the ordinarily skilled artisan, any ZF protein and its cognate ZF binding sequence may be used as a nuclear targeting factor (NTF) and cognate TFBS in the compositions and methods of the present disclosure. In some embodiments the DTS comprises a ZF-responsive TFBS having a sequence identity of 90% or more to AAACTGCAAAAG.

In some embodiments, the TFBS is a binding sequence for a TAL effector DNA-binding domain-containing protein (“TALE protein”). A TALE is a protein of 32 fixed amino acids and 2 variable residues, the 2 residues being engineered to recognize specific nucleotides (e.g., NN for G, NI for A, HD for C, etc). As will be appreciated by the ordinarily skilled artisan, any TALE protein and its cognate TALE binding sequence may be used in the compositions and methods of the present disclosure, such examples being found in, e.g., Li et al. 2011 (Modularly assembled designer TAL effector nucleases for targeted gene knockout and gene replacement in eukaryotes. Nucleic Acids Research, Volume 39, Issue 14, pp 6315-6325) and Kim et. al. 2013 (A library of TAL effector nucleases spanning the human genome. Nature Biotechnology volume 31, pages 251-258 (2013). In some embodiments, the TALE TFBS has 90% identity or more to a sequence is selected from the group consisting of TTCATTACACCTGCAGCT (SEQ ID NO: 144), ATAAACCCCCTCCAA (SEQ ID NO:145), and TCGAGTTTACTCCCTATCAGTGATAGAGAACG (SEQ ID NO:146).

In some embodiments, the TFBS is a binding sequence for a GAL4 protein. In some such embodiments, the GAL4 TFBS comprises the sequence CGG-N11-CCG, for example, CGGAGGACTGTCCTCCG (SEQ ID NO:147). In some embodiments, the DTS comprises 2, 3, 4, 5, 6, 7, 8, 9, or 10 copies of the GAL4 TFBS. In certain embodiments, the DTS comprises 5 copies of the GAL4 TFBS. In certain embodiments, the DTS comprises 7 copies of the GAL4 TFBS. In certain embodiments, the DTS comprises 10 or more copies of the GAL4 TFBS. In certain embodiments, the DTS comprises an upstream activation sequence (UAS) for the native GAL4 protein, as known in the art. In certain embodiments, the DTS consists essentially of a UAS. In some embodiments, the DTS comprises a sequence having 80% identity or more to a sequence listed in Table 5, for example, 85% or 90% identity or more, in some cases 95% identity or more, e.g., 96%, 97%, 98%, or 99% identity to a sequence in Table 5. In some instances, the DTS is identical to a sequence in Table 5.

TABLE 5
Examples of DTSs comprising a GAL4 TFBS (e.g., UAS)

| DTS | Sequence (5′ to 3′) |
|---|---|
| UAS Motif | CGG-N₁₁-CCG (SEQ ID NO: 148) |
| UAS Core A | CGGTGGCTTCTAATCCG (SEQ ID NO: 149) |
| UAS Core B | CGGGTGACAGCCCTCCG (SEQ ID NO: 150) |
| UAS Core C | CGGAGGAGAGTCTTCCG (SEQ ID NO: 151) |
| UAS Core D | CGGAGTACTGTCCTCCG (SEQ ID NO: 152) |
| UAS Core E | CGGAGGACTGTCCTCCG (SEQ ID NO: 153) |
| UAS-1 | ACACTAGAGTCGGAGGACTGTCCTCCGTGAGTCCTAG (SEQ ID NO: 154) |
| UAS-2 | CGGAGGACTGTCCTCCGTGAGTCCTAGCGGAGGACTGTCCTCCG (SEQ ID |
| | NO: 155) |
| UAS-3 | CGGAGGACTGTCCTCCGTGAGTCCTAGCGGAGGACTGTCCTCCGTCTTCAC |
| | AGGCGGAGGACTGTCCTCCG (SEQ ID NO: 156) |
| UAS-4 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCG (SEQ |
| | ID NO: 157) |
| UAS-5 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCGACAC |
| | TAGAGTCGGAGGACTGTCCTCCG (SEQ ID NO: 158) |
| UAS-5-NR | CGGTGGCTTCTAATCCGTGAGTCCTAGCGGGTGACAGCCCTCCGTCTTCAC |
| | AGGCGGAGGAGAGTCTTCCGTAGGGTTCCTCGGAGTACTGTCCTCCGACAC |
| | TAGAGTCGGAGGACTGTCCTCCG (SEQ ID NO: 159) |
| UAS-6 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCGACAC |
| | TAGAGTCGGAGGACTGTCCTCCGGATAGTATCACGGAGGACTGTCCTCCG |
| | (SEQ ID NO: 160) |
| UAS-7 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCGACAC |
| | TAGAGTCGGAGGACTGTCCTCCGGATAGTATCACGGAGGACTGTCCTCCGA |
| | GCAAACGAACGGAGGACTGTCCTCCG (SEQ ID NO: 161) |
| UAS-10 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCGACAC |
| | TAGAGTCGGAGGACTGTCCTCCGGATAGTATCACGGAGGACTGTCCTCCGA |
| | ATGGACTAACGGAGGACTGTCCTCCGAGCAAACGAACGGAGGACTGTCCTC |
| | CGTACACCGATCCGGAGGACTGTCCTCCGGCGCTCGGCACGGAGGACTGT |
| | CCTCCG (SEQ ID NO: 162) |
| UAS-20 | CGGAGGACTGTCCTCCGTGAGTCCTAGCGGAGGACTGTCCTCCGTCTTCAC |
| | AGGCGGAGGACTGTCCTCCGTAGGGTTCCTCGGAGGACTGTCCTCCGACAC |
| | TAGAGTCGGAGGACTGTCCTCCGGATAGTATCACGGAGGACTGTCCTCCGA |
| | ATGGACTAACGGAGGACTGTCCTCCGACTTTAGCGCCGGAGGACTGTCCTC |
| | CGTACACCGATCCGGAGGACTGTCCTCCGGCGCTCGGCACGGAGGACTGT |
| | CCTCCGCGAAAACGGGCGGAGGACTGTCCTCCGTGCGTGGATTCGGAGGA |
| | CTGTCCTCCGAGCCAGGAGCCGGAGGACTGTCCTCCGAAGACCACGGCGG |
| | AGGACTGTCCTCCGACCAGTACCCCGGAGGACTGTCCTCCGATGTCTTGTA |
| | CGGAGGACTGTCCTCCGCTGACTATCGCGGAGGACTGTCCTCCGCCTACGT |
| | CATCGGAGGACTGTCCTCCGATAGAAACCTCGGAGGACTGTCCTCCGCATG |
| | TCTAACCGGAGGACTGTCCTCCG (SEQ ID NO: 163) |

In some embodiments, the TFBS is a binding sequence for an Arc protein, where Arc is a bacteriophage regulatory protein. In some such embodiments, the Arc TFBS comprises the sequence RYRVTAGANNNNNTCTABYRY (SEQ ID NO:164), for example, ATGATAGAAGCACTCTACTAT (SEQ ID NO:165). In some embodiments, the Arc TFBS comprises a sequence having 80%, 85%, 90% identity or more to ATGATAGAAGCACTCTACTAT (SEQ ID NO:166). In some embodiments, the DTS comprises 2, 3, 4, 5, 6, 7, 8, 9, or 10 copies of the Arc TFBS. In certain embodiments, the DTS comprises 5 copies of the Arc TFBS. In certain embodiments, the DTS comprises 7 copies of the Arc TFBS. In certain embodiments, the DTS comprises 10 or more copies of the Arc TFBS. In some embodiments, the DTS comprises a sequence having 80%, 85%, or 90% identity or more to the sequence
ATGATAGAAGCACTCTACTATTGAGTCCTAGATGATAGAAGCACTCTACTATTCTTCACAGG ATGATAGAAGCACTCTACTATTAGGGTTCCTATGATAGAAGCACTCTACTATACACTAGAGT ATGATAGAAGCACTCTACTATGATAGTATCAATGATAGAAGCACTCTACTATAGCAAACGAA ATGATAGAAGCACTCTACTAT (SEQ ID NO:167), in some cases 95% identity or more to this sequence, in certain cases sharing 100% identity with this sequence.

In some embodiments, the TFBS is a binding sequence for a Mnt protein, where Mnt is a bacteriophage regulatory protein. In some such embodiments, the Mnt TFBS comprises the sequence GGNCCACNGTGGNCC (SEQ ID NO:168), for example,
ATAGGTCCACGGTGGACCATA (SEQ ID NO:169). In some embodiments, the Mnt TFBS comprises a sequence having 80%, 85%, 90% identity or more to ATAGGTCCACGGTGGACCATA (SEQ ID NO:170). In some embodiments, the DTS comprises 2, 3, 4, 5, 6, 7, 8, 9, or 10 copies of the Mnt TFBS. In certain embodiments, the DTS comprises 5 copies of the Mnt TFBS. In certain embodiments, the DTS comprises 7 copies of the Mnt TFBS. In certain embodiments, the DTS comprises 10 or more copies of the Mnt TFBS. In some embodiments, the DTS comprises a sequence having 80%, 85%, or 90% identity or more to the
sequenceATAGGTCCACGGTGGACCATATGAGTCCTAGATAGGTCCACGGTGGACCATATC TTCACAGGATAGGTCCACGGTGGACCATATAGGGTTCCTATAGGTCCACGGTGGACCATA ACACTAGAGTATAGGTCCACGGTGGACCATAGATAGTATCAATAGGTCCACGGTGGACCAT AAGCAAACGAAATAGGTCCACGGTGGACCATA (SEQ ID NO:171), in some cases 95% identity or more to this sequence, in certain cases sharing 100% identity with this sequence.

In some embodiments, the TFBS is a binding sequence for a purine synthesis repressor (PurR) protein. In some such embodiments, the PurR TFBS comprises a sequence having 80%, 85%, 90% identity or more to ACGCAAACGTTTTCGT (SEQ ID NO:172), in some cases 95% identity or more to this sequence, in certain cases sharing 100% identity with this sequence. In some embodiments, the DTS comprises 2, 3, 4, 5, 6, 7, 8, 9, or 10 copies of the PurR TFBS. In certain embodiments, the DTS comprises 5 copies of the PurR TFBS. In certain embodiments, the DTS comprises 7 copies of the PurR TFBS. In certain embodiments, the DTS comprises 10 or more copies of the PurR TFBS. I In some embodiments, the DTS comprises a sequence having 80%, 85%, or 90% identity or more to the sequence
ACGCAAACGTTTTCGTTGAGTCCTAGACGCAAACGTTTTCGTTCTTCACAGGACGCAAACG TTTTCGTTAGGGTTCCTACGCAAACGTTTTCGTACACTAGAGTACGCAAACGTTTTCGTGAT AGTATCAACGCAAACGTTTTCGTAGCAAACGAAACGCAAACGTTTTCGT (SEQ ID NO:173), in some cases 95% identity or more to this sequence, in certain cases sharing 100% identity with this sequence.

In some embodiments, the TFBS is a binding sequence for a Bac434 protein, where Bac434 is a bacteriophage regulatory protein. In some such embodiments, the Bac434 TFBS comprises a sequence that has 80%, 85%, 90% identity or more to ACAAGAAAGTTTGT (SEQ ID NO: 174), ACAAGATACATTGT (SEQ ID NO:175), or ACAAGAAAAACTGT (SEQ ID NO: 176), in some cases 95% identity or more to ACAAGAAAGTTTGT (SEQ ID NO:177), ACAAGATACATTGT (SEQ ID NO:178), or ACAAGAAAAACTGT (SEQ ID NO:179), in certain cases sharing 100% identity with ACAAGAAAGTTTGT (SEQ ID NO:180), ACAAGATACATTGT (SEQ ID NO:181), or ACAAGAAAAACTGT (SEQ ID NO:182). In some embodiments, the DTS comprises 2, 3, 4, 5, 6, 7, 8, 9, or 10 copies of the Bac434 TFBS. In certain embodiments, the DTS comprises 5 copies of the Bac434 TFBS. In certain embodiments, the DTS comprises 7 copies of the Bac434 TFBS. In certain embodiments, the DTS comprises 10 or more copies of the Bac434 TFBS. In certain embodiments, the DTS comprises a sequence having 80%, 85%, or 90% identity or more to a sequence listed in Table 6, for example, 85% or 90% identity or more, in some cases 95% identity or more, e.g. 96%, 97%, 98%, or 99% identity to a sequence in Table 6. In some instances, the DTS is identical to a sequence in Table 6.

TABLE 6
Examples of DTSs comprising a Bac434 TFBS

| DTS | Sequence (5′ to 3′) |
|---|---|
| Bac434-OR1-Core | ACAAGAAAGTTTGT (SEQ ID NO: 183) |
| Bac434-OR2-Core | ACAAGATACATTGT (SEQ ID NO: 184) |
| Bac434-OR3-Core | ACAAGAAAAACTGT (SEQ ID NO: 185) |
| Bac434-OR321- | ACAAGAAAAACTGTATTTGACAAACAAGATACATTGTATGAAAATACAA |
| Core | GAAAGTTTGT (SEQ ID NO: 186) |
| Bac434-OR1-7 | ACAAGAAAGTTTGTTGAGTCCTAGACAAGAAAGTTTGTTCTTCACAGGA |
| | CAAGAAAGTTTGTTAGGGTTCCTACAAGAAAGTTTGTACACTAGAGTAC |
| | AAGAAAGTTTGTGATAGTATCAACAAGAAAGTTTGTAGCAAACGAAACA |
| | AGAAAGTTTGT (SEQ ID NO: 187) |
| Bac434-OR3-7 | ACAAGAAAAACTGTTGAGTCCTAGACAAGAAAAACTGTTCTTCACAGGA |
| | CAAGAAAAACTGTTAGGGTTCCTACAAGAAAAACTGTACACTAGAGTAC |
| | AAGAAAAACTGTGATAGTATCAACAAGAAAAACTGTAGCAAACGAAACA |
| | AGAAAAACTGT (SEQ ID NO: 188) |
| Bac434-OR321-3 | CCCAATCTTCACAAGAAAAACTGTATTTGACAAACAAGATACATTGTAT |
| | GAAAATACAAGAAAGTTTGTTGATGGAGGCACAAGAAAAACTGTATTTG |
| | ACAAACAAGATACATTGTATGAAAATACAAGAAAGTTTGTTGATGGAGG |
| | CACAAGAAAAACTGTATTTGACAAACAAGATACATTGTATGAAAATACA |
| | AGAAAGTTTGTTGATGGAGGC (SEQ ID NO: 189) |

In some embodiments, the TFBS is a binding sequence for a GCN4 protein. In some such embodiments, the GCN4 TFBS comprises a sequence having 80%, 85%, 90% identity or more to TGACTC (SEQ ID NO:190), in some cases 95% identity or more to TGACTC (SEQ ID NO: 190), in certain cases 100% identity with TGACTC (190), for example, AGTGACTCATT (SEQ ID NO:191). In some embodiments, the DTS comprises 2, 3, 4, 5, 6, 7, 8, 9, or 10 copies of the GCN4 TFBS. In certain embodiments, the DTS comprises 5 copies of the GCN4 TFBS. In certain embodiments, the DTS comprises 7 copies of the GCN4 TFBS. In certain embodiments, the DTS comprises 10 or more copies of the GCN4 TFBS. In some embodiments, the DTS comprises comprises a sequence having 80%, 85%, or 90% identity or more to the sequence
AGTGACTCATTTGAGTCCTAGAGTGACTCATTTCTTCACAGGAGTGACTCATTTAGGGTTCC TAGTGACTCATTACACTAGAGTAGTGACTCATTGATAGTATCAAGTGACTCATTAGCAAACG AAAGTGACTCATT (192), in some cases 95% identity or more to this sequence, in certain cases sharing 100% identity with this sequence.

In some embodiments, the TFBS is a binding sequence for a Lactose Inhibitor (Lacl) protein, also referred to herein as a Lactose Repressor (LacR) protein, for example, a LacO sequence, e.g., TTGTTATCCGCTCACAA (SEQ ID NO:193). In some such embodiments, the LacR TFBS comprises a sequence having 80%, 85%, 90% identity or more to TTGTTATCCGCTCACAA (SEQ ID NO:193). In certain embodiments, the DTS comprises a lactose operon (LacO), as known in the art. In certain embodiments, the DTS consists essentially of a LacO. In some embodiments, the DTS comprises 2, 3, 4, 5, 6, 7, 8, 9, or 10 copies of the LacR TFBS. In certain embodiments, the DTS comprises 5 copies of the LacR TFBS. In certain embodiments, the DTS comprises 7 copies of the LacR TFBS. In certain embodiments, the DTS comprises 10 or more copies of the LacR TFBS. In some embodiments, the DTS comprises a sequence that has 80%, 85%, or 90% identity or more to the sequence TTGTTATCCGCTCACAATTCCACATGTGGCCACAAATTGTTATCCGCTCACAATTCCACATG TGGCCACAAATTGTTATCCGCTCACAATTCCACATGTGGCCACAAATTGTTATCCGCTCACA ATTCCACATGTGGCCACAAATTGTTATCCGCTCACAATTCCACATGTGGCCACAAATTGTTA TCCGCTCACAATTCCACATGTGGCCACAAATTGTTATCCGCTCACAA (SEQ ID NO:194), in some cases 95% identity or more to this sequence, in certain cases sharing 100% identity with this sequence.

In some embodiments, the TFBS is a binding sequence for an endonuclease, e.g., a I-Scel D44A protein. In some embodiments the DTS comprises a TFBS having 90% identity or more to the sequence TAGGGATAACAGGGTAAT (SEQ ID NO:195). Accordingly, in some instances, the auxiliary RNA may include coding sequence that encodes the DNA-binding domain of the endonuclease corresponding to the sequence bound by the endonuclease that is employed as the TFBS.

As another example, when the coding sequence encodes a DNA binding domain of a I-Scel protein, the Scel protein may include a sequence having 85% identity or more to MKNIKKNQVMNLGPNSKLLKEYKSQLIELNIEQFEAGIGLILGAAYIRSRDEGKTYCMQFEWKNK AYMDHVCLLYDQWVLSPPHKKERVNHLGNLVITWGAQTFKHQAFNKLANLFIVNNKKTIPNNLV ENYLTPMSLAYWFMDDGGKWDYNKNSTNKSIVLNTQSFTFEEVEYLVKGLRNKFQLNCYVKIN KNKPIIYIDSMSYLIFYNLIKPYLIPQMMYKLPNTISSETFLK (SEQ ID NO:196), e.g., a sequence having 85% identity more to
MPKKKRKVPKKHAAPPKKKRKVEDPRFMYPYDVPDYAGMKNIKKNQVMNLGPNSKLLKEYKS QLIELNIEQFEAGIGLILGAAYIRSRDEGKTYCMQFEWKNKAYMDHVCLLYDQWVLSPPHKKER VNHLGNLVITWGAQTFKHQAFNKLANLFIVNNKKTIPNNLVENYLTPMSLAYWFMDDGGKWDY NKNSTNKSIVLNTQSFTFEEVEYLVKGLRNKFQLNCYVKINKNKPIIYIDSMSYLIFYNLIKPYLIPQ MMYKLPNTISSETFLK (SEQ ID NO:197). In some embodiments, the I-Scel protein has been engineered to comprise an NLS. In some embodiments, the NLS is proximal to the N terminus. In other embodiments, the NLS is proximal to the C-terminus. In some embodiments, the I-Scel protein is engineered to comprise an NLS at both the N-terminus and the C-terminus. In some embodiments, the NLS is fused to the I-Scel protein with a linker.

It will be appreciated by one of ordinary skill in the art that DNA binding proteins can tolerate some degree of nucleotide substitution in their binding sequences and that the TFBSs provided herein are but examples of sequences that may be used. In some embodiments, the TFBS shares 80% identity or more with a sequence disclosed herein, for example, 85%, 90%, 95% identity or more, e.g., 96%, 97%, 98%, or 99% identity or more, in certain instances 100% identity to the sequence. Publicly available databases such as Uniprot, Cis-BP, Transfac, and GrassiusX can be consulted to identify which nucleotides can be varied and which should be conserved in designing sequences for use in the systems, compositions, and methods of the present disclosure.

### Genomic Integration and Editing

In some instances, a gene editing system, e.g., that is configured to provide genomic integration of payload DNA or a donor template component of the payload DNA, is employed. Gene editing systems that may be employed in such embodiments may vary, as desired. In some embodiments, the employed gene editing system is configured to genomically integrate the payload DNA, or portion thereof, into a specific safe harbor location in the genome, for example to a genomic location within or near an endogenous albumin locus. Generally, one skilled in the art will understand that a safe harbor locus is a location within a genome that can be used for integrating exogenous nucleic acids, where the addition of exogenous nucleic acids into the safe harbor locus does not cause significant effect on the growth of the host cell by the addition of the nucleic acids alone. In some embodiments, the payload DNA or donor template may be inserted into the specific safe harbor location in the genome that may either utilize the promoter found at that safe harbor locus, or allow the expressional regulation of a coding sequence of the payload DNA or donor template by an exogenous promoter that is fused to the payload DNA or donor template coding sequence prior to insertion.

Gene editing can be conducted using nucleases engineered to target specific sequences. To date there are four major types of nucleases: meganucleases and their derivatives, zinc finger nucleases (ZFNs), transcription activator like effector nucleases (TALENs), and CRISPR-Cas9 nuclease systems. The nuclease platforms vary in difficulty of design, targeting density and mode of action, particularly as the specificity of ZFNs and TALENs is through protein-DNA interactions, while RNA-DNA interactions primarily guide Cas9. Cas9 cleavage also requires an adjacent motif, the PAM, which differs between different CRISPR systems. Cas9 from Streptococcus pyogenes cleaves using a NRG PAM, CRISPR from Neisseria meningitidis can cleave at sites with PAMs including NNNNGATT, NNNNNGTTT and NNNNGCTT. A number of other Cas9 orthologs target protospacer adjacent to alternative PAMs. CRISPR endonucleases, such as Cas9, can be used in various embodiments of the methods of the disclosure. However, the teachings described herein, such as therapeutic target sites, could be applied to other forms of endonucleases, such as ZFNs, TALENs, HEs, or MegaTALs, or using combinations of nucleases. These different systems are now described in further detail.

A given LNP system of the present disclosure may include a one or more elements of a given gene editing system. Gene editing system employed in embodiments of the invention may include a number of different elements, such as nucleic acid elements, e.g., genome-targeting nucleic acids or Guide RNAs, nucleic acids, e.g., mRNAs encoding endonucleases; polypeptide components, e.g., endonucleases, etc. These various components are now reviewed in greater detail in conjunction with the description of representative endonuclease based genomic integration systems.

In some embodiments, the methods of genome editing and compositions therefore use a nucleic acid sequence (or oligonucleotide) encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid into genomic DNA is an example of genome editing. A site-directed polypeptide is a nuclease used in genome editing to cleave DNA. The site-directed can be administered to a cell or a patient as either: one or more polypeptides, or one or more mRNAs encoding the polypeptide. In some embodiments, a site-directed polypeptide has a plurality of nucleic acid-cleaving (i.e., nuclease) domains. Two or more nucleic acid-cleaving domains can be linked together via a linker. In some embodiments, the linker has a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

CRISPR. In the context of a CRISPR/Cas or CRISPR/Cpf1 system, the site-directed polypeptide can bind to a guide RNA that, in turn, specifies the site in the target DNA to which the polypeptide is directed. In some embodiments of CRISPR/Cas or CRISPR/Cpf1 systems herein, the site-directed polypeptide is an endonuclease, such as a DNA endonuclease.

Naturally-occurring wild-type Cas9 enzymes have two nuclease domains, a HNH nuclease domain and a RuvC domain. Herein, the “Cas9” refers to both naturally-occurring and recombinant Cas9s. Cas9 enzymes contemplated herein have a HNH or HNH-like nuclease domain, and/or a RuvC or RuvC-like nuclease domain.

HNH or HNH-like domains have a McrA-like fold. HNH or HNH-like domains has two antiparallel beta-strands and an alpha-helix. HNH or HNH-like domains has a metal binding site (e.g., a divalent cation binding site). HNH or HNH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 beta-strands surrounded by a plurality of alpha-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide (e.g., Cas9 from S. pyogenes, US2014/0068797 Sequence ID No. 8 or Sapranauskas et al., Nucleic Acids Res, 39 (21): 9275-9282 (2011)), and various other site-directed polypeptides). In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra). In some embodiments, a site-directed polypeptide has at least 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

In some embodiments, the site-directed polypeptide has a modified form of a wild-type exemplary site-directed polypeptide. The modified form of the wild-type exemplary site-directed polypeptide has a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide has a mutation such that it can induce a single-strand break (SSB) on a target nucleic acid (e.g., by cutting only one of the sugar-phosphate backbones of a double-strand target nucleic acid). In some embodiments, the mutation results in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). In some embodiments, the mutation results in one or more of the plurality of nucleic acid-cleaving domains retaining the ability to cleave the complementary strand of the target nucleic acid, but reducing its ability to cleave the non-complementary strand of the target nucleic acid. In some embodiments, the mutation results in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854 and Asn856, are mutated to inactivate one or more of the plurality of nucleic acid-cleaving domains (e.g., nuclease domains). In some embodiments, the residues to be mutated correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. One skilled in the art will recognize that mutations other than alanine substitutions are suitable.

In some embodiments, a D10A mutation is combined with one or more of H840A, N854A, or N856A mutations to produce a site-directed polypeptide substantially lacking DNA cleavage activity. In some embodiments, a H840A mutation is combined with one or more of D10A, N854A, or N856A mutations to produce a site-directed polypeptide substantially lacking DNA cleavage activity. In some embodiments, a N854A mutation is combined with one or more of H840A, D10A, or N856A mutations to produce a site-directed polypeptide substantially lacking DNA cleavage activity. In some embodiments, a N856A mutation is combined with one or more of H840A, N854A, or D10A mutations to produce a site-directed polypeptide substantially lacking DNA cleavage activity. Site-directed polypeptides that have one substantially inactive nuclease domain are referred to as “nickases”.

In some embodiments, variants of RNA-guided endonucleases, for example Cas9, can be used to increase the specificity of CRISPR-mediated genome editing. Wild type Cas9 is generally guided by a single guide RNA designed to hybridize with a specified about 20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes. Descriptions of various CRISPR/Cas systems for use in gene editing can be found, e.g., in international patent application publication number WO2013/176772, and in Nature Biotechnology 32, 347-355 (2014), and references cited therein.

In some embodiments, the site-directed polypeptide (e.g., variant, mutated, enzymatically inactive and/or conditionally enzymatically inactive site-directed polypeptide) targets nucleic acid. In some embodiments, the site-directed polypeptide (e.g., variant, mutated, enzymatically inactive and/or conditionally enzymatically inactive endoribonuclease) targets DNA. In some embodiments, the site-directed polypeptide (e.g., variant, mutated, enzymatically inactive and/or conditionally enzymatically inactive endoribonuclease) targets RNA.

In some embodiments, the site-directed polypeptide has one or more non-native sequences (e.g., the site-directed polypeptide is a fusion protein). In some embodiments, the site-directed polypeptide has an amino acid sequence having at least 15% amino acid identity to a Cas9 from a bacterium (e.g., S. pyogenes), a nucleic acid binding domain, and two nucleic acid cleaving domains (i.e., a HNH domain and a RuvC domain). In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain). In some embodiments, the site-directed polypeptide has an amino acid sequence having at least 15% amino acid identity to a Cas9 from a bacterium (e.g., S. pyogenes), and two nucleic acid cleaving domains, wherein one or both of the nucleic acid cleaving domains have at least 50% amino acid identity to a nuclease domain from Cas9 from a bacterium (e.g., S. pyogenes). In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), and non-native sequence (for example, a nuclear localization signal) or a linker linking the site-directed polypeptide to a non-native sequence. In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), wherein the site-directed polypeptide has a mutation in one or both of the nucleic acid cleaving domains that reduces the cleaving activity of the nuclease domains by at least 50%. In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain), wherein one of the nuclease domains has mutation of aspartic acid 10, and/or wherein one of the nuclease domains has mutation of histidine 840, and wherein the mutation reduces the cleaving activity of the nuclease domain(s) by at least 50%.

In some embodiments, the one or more site-directed polypeptides, e.g., DNA endonucleases, include two nickases that together effect one double-strand break at a specific locus in the genome, or four nickases that together effect two double-strand breaks at specific loci in the genome. Alternatively, one site-directed polypeptide, e.g., DNA endonuclease, affects one double-strand break at a specific locus in the genome.

In some embodiments, a polynucleotide encoding a site-directed polypeptide can be used to edit genome. In some of such embodiments, the polynucleotide encoding a site-directed polypeptide is codon-optimized according to methods standard in the art for expression in the cell containing the target DNA of interest. For example, if the intended target nucleic acid is in a human cell, a human codon-optimized polynucleotide encoding Cas9 is contemplated for use for producing the Cas9 polypeptide.

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary hairpin structures (e.g., hairpins) and/or have unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

crRNA biogenesis in a Type II CRISPR system in nature requires a trans-activating CRISPR RNA (tracrRNA). The tracrRNA is modified by endogenous RNaselll, and then hybridizes to a crRNA repeat in the pre-crRNA array. Endogenous RNaselll is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337 (6096): 816-821 (2012) showed that the CRISPR/Cas9 system is useful for RNA-programmable genome editing, and international patent application publication number WO 2013/176772 provides numerous examples and applications of the CRISPR/Cas endonuclease system for site-specific gene editing.

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAS without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

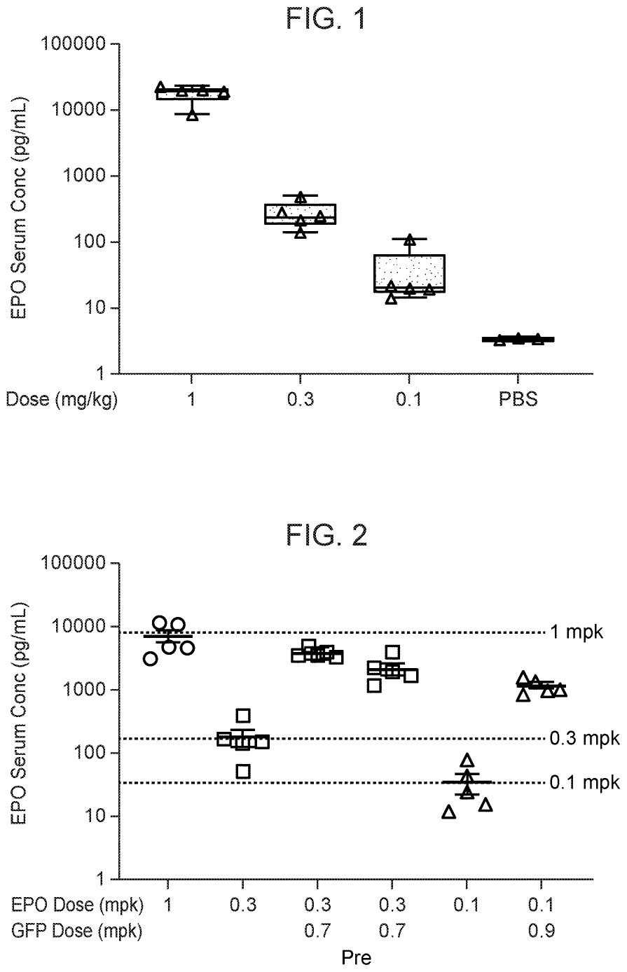

Exemplary CRISPR/Cas polypeptides include the Cas9 polypeptides in FIG. 1 of Fonfara et al., Nucleic Acids Research, 42:2577-2590 (2014). The CRISPR/Cas gene naming system has undergone extensive rewriting since the Cas genes were discovered.

A genome-targeting nucleic acid interacts with a site-directed polypeptide (e.g., a nucleic acid-guided nuclease such as Cas9), thereby forming a complex. The genome-targeting nucleic acid (e.g., gRNA, such as described in greater detail below) guides the site-directed polypeptide to a target nucleic acid.

In some embodiments the site-directed polypeptide and genome-targeting nucleic acid can each be administered separately to a cell or a patient. On the other hand, in some other embodiments the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

Genome-Targeting Nucleic Acid or Guide RNA. Genomic editing components may include a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC), which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments of genome-targeting nucleic acids, a spacer extension sequence can modify activity, provide stability and/or provide a location for modifications of a genome-targeting nucleic acid. A spacer extension sequence can modify on- or off-target activity or specificity. In some embodiments, a spacer extension sequence is provided. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of about 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. In some embodiments, a spacer extension sequence is less than 10 nucleotides in length. In some embodiments, a spacer extension sequence is between 10-30 nucleotides in length. In some embodiments, a spacer extension sequence is between 30-70 nucleotides in length.

In some embodiments, the spacer extension sequence has another moiety (e.g., a stability control sequence, an endoribonuclease binding sequence, a ribozyme). In some embodiments, the moiety decreases or increases the stability of a nucleic acid targeting nucleic acid. In some embodiments, the moiety is a transcriptional terminator segment (i.e., a transcription termination sequence). In some embodiments, the moiety functions in a eukaryotic cell. In some embodiments, the moiety functions in a prokaryotic cell. In some embodiments, the moiety functions in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

The spacer sequence hybridizes to a sequence in a target nucleic acid of interest. The spacer of a genome-targeting nucleic acid interacts with a target nucleic acid in a sequence-specific manner via hybridization (i.e., base pairing). The nucleotide sequence of the spacer thus varies depending on the sequence of the target nucleic acid of interest.

In a CRISPR/Cas system herein, the spacer sequence is designed to hybridize to a target nucleic acid that is located 5′ of a PAM of the Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

In some embodiments, the target nucleic acid sequence has 20 nucleotides. In some embodiments, the target nucleic acid has less than 20 nucleotides. In some embodiments, the target nucleic acid has more than 20 nucleotides. In some embodiments, the target nucleic acid has at least: 5, 10, 15, 16, 17, 18, 19, 20, 21, 22, 23, 24, 25, 30 or more nucleotides. In some embodiments, the target nucleic acid has at most: 5, 10, 15, 16, 17, 18, 19, 20, 21, 22, 23, 24, 25, 30 or more nucleotides. In some embodiments, the target nucleic acid sequence has 20 bases immediately 5′ of the first nucleotide of the PAM. For example, in a sequence having 5′-NNNNNNNNNNNNNNNNNNNNNRG-3′, the target nucleic acid has the sequence that corresponds to the Ns, wherein N is any nucleotide, and the underlined NRG sequence (R is G or A) is the Streptococcus pyogenes Cas9 PAM. In some embodiments, the PAM sequence used in the compositions and methods of the present disclosure as a sequence recognized by S.p. Cas9 is NGG.

In some embodiments, the spacer sequence that hybridizes to the target nucleic acid has a length of at least about 6 nucleotides (nt). The spacer sequence can be at least about 6 nt, about 10 nt, about 15 nt, about 18 nt, about 19 nt, about 20 nt, about 25 nt, about 30 nt, about 35 nt or about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some embodiments, the spacer sequence has 20 nucleotides. In some embodiments, the spacer has 19 nucleotides. In some embodiments, the spacer has 18 nucleotides. In some embodiments, the spacer has 17 nucleotides. In some embodiments, the spacer has 16 nucleotides. In some embodiments, the spacer has 15 nucleotides.

In some embodiments, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some embodiments, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In some embodiments, the length of the spacer sequence and the target nucleic acid can differ by 1 to 6 nucleotides, which can be thought of as a bulge or bulges.

In some embodiments, the spacer sequence is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

Zinc Finger Nucleases. Zinc finger nucleases (ZFNs) are modular proteins having an engineered zinc finger DNA binding domain linked to the catalytic domain of the type II endonuclease Fokl. Because Fokl functions only as a dimer, a pair of ZFNs must be engineered to bind to cognate target “half-site” sequences on opposite DNA strands and with precise spacing between them to enable the catalytically active Fokl dimer to form. Upon dimerization of the Fokl domain, which itself has no sequence specificity per se, a DNA double-strand break is generated between the ZFN half-sites as the initiating step in genome editing.

The DNA binding domain of each ZFN generally has 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers, although considerable expertise is required to do this well. In most applications of ZFNs, proteins of 4-6 fingers are used, recognizing 12-18 bp respectively. Hence, a pair of ZFNs will generally recognize a combined target sequence of 24-36 bp, not including the 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the Fokl domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these Fokl variants.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96(6):2758-63 (1999); Dreier B et al., J Mol Biol. 303(4):489-502 (2000); Liu Q et al., J Biol Chem. 277(6):3850-6 (2002); Dreier et al., J Biol Chem 280(42):35588-97 (2005); and Dreier et al., J Biol Chem. 276(31):29466-78 (2001).

Transcription Activator-Like Effector Nucleases (TALENs). TALENs represent another format of modular nucleases whereby, as with ZFNs, an engineered DNA binding domain is linked to the Fokl nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs have tandem arrays of 33-35 amino acid repeats, with each repeat recognizing a single base pair in the target DNA sequence that is generally up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the Fokl domain to reduce off-target activity.

Additional variants of the Fokl domain have been created that are deactivated in their catalytic function. If one half of either a TALEN or a ZFN pair contains an inactive Fokl domain, then only single-strand DNA cleavage (nicking) will occur at the target site, rather than a DSB. The outcome is comparable to the use of CRISPR/Cas9/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science 326(5959):1509-12 (2009); Mak et al., Science 335(6069):716-9 (2012); and Moscou et al., Science 326 (5959): 1501 (2009). The use of TALENs based on the “Golden Gate” platform, or cloning scheme, has been described by multiple groups; see, e.g., Cermak et al., Nucleic Acids Res. 39(12):e82 (2011); Li et al., Nucleic Acids Res. 39(14):6315-25 (2011); Weber et al., PLOS One. 6(2):e16765 (2011); Wang et al., J Genet Genomics 41(6):339-47, Epub 2014 Can 17 (2014); and Cermak T et al., Methods Mol Biol. 1239:133-59 (2015).

Homing Endonucleases. Homing endonucleases (HEs) are sequence-specific endonucleases that have long recognition sequences (14-44 base pairs) and cleave DNA with high specificity—often at sites unique in the genome. There are at least six known families of HEs as classified by their structure, including LAGLIDADG (SEQ ID NO:6), GIY-YIG, His-Cis box, H-N-H, PD-(D/E) xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24(8):663-80 (2014); Belfort and Bonocora, Methods Mol Biol. 1123:1-26 (2014); Hafez and Hausner, Genome 55(8):553-69 (2012); and references cited therein.

MegaTAL/Tev-mTALEN/MegaTev. As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., NAR 42:2591-2601 (2014); Kleinstiver et al., G3 4:1155-65 (2014); and Boissel and Scharenberg, Methods Mol. Biol. 1239:171-96 (2015).

In a further variation, the MegaTev architecture is the fusion of a meganuclease (Mega) with the nuclease domain derived from the GIY-YIG homing endonuclease I-Tevl (Tev). The two active sites are positioned about30 bp apart on a DNA substrate and generate two DSBs with non-compatible cohesive ends; see, e.g., Wolfs et al., NAR 42, 8816-29 (2014). It is anticipated that other combinations of existing nuclease-based approaches will evolve and be useful in achieving the targeted genome modifications described herein.

dCas9-Fokl or dCpf1-Fokl and Other Nucleases. Combining the structural and functional properties of the nuclease platforms described above offers a further approach to genome editing that can potentially overcome some of the inherent deficiencies. As an example, the CRISPR genome editing system generally uses a single Cas9 endonuclease to create a DSB. The specificity of targeting is driven by a 20 or 22 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a Fokl domain to the deactivated Cas9; see, e.g., Tsai et al., Nature Biotech 32:569-76 (2014); and Guilinger et al., Nature Biotech. 32:577-82 (2014). Because Fokl must dimerize to become catalytically active, two guide RNAs are required to tether two Fokl fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-Tevl, takes advantage of both the tunable DNA binding and specificity of the TALE, as well as the cleavage sequence specificity of I-Tevl, with the expectation that off-target cleavage can be further reduced.

Additional details regarding gene editing systems that find use in embodiments of the invention may be found in United States Published Patent Application Publication No. 20210348159.

### Additional Components

In some embodiments, the compositions and systems of the present disclosure may include one or more additional components. For example, an LNP system or LNP composition may include one or more additional components, i.e., in addition to the LNP, payload DNA, and auxiliary RNA components, such as e.g., one or more compounds, including compounds that are not a nucleic acid or component of an LNP.

The one or more additional compounds can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the payload DNA within the LNP is useful for treating cancer, the additional compound can be an anti-cancer agent (e.g., a chemotherapeutic agent, an immune-oncology agent (e.g., an immune checkpoint inhibitor), a targeted cancer therapy (including, but not limited to, a small molecule, an antibody, or an antibody-drug conjugate). In another example, if the LNP containing the payload DNA is useful for treating an infection, the additional compound can be an antimicrobial agent (e.g., an antibiotic or antiviral compound). In yet another example, if the LNP containing the payload DNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a payload DNA encoding a different protein or a different compound, such as a therapeutic may be used in the compositions and methods of the invention. In some embodiments, the additional compound is an immune modulating agent. For example, the additional compound is an immunosuppressant. In some embodiments, the additional compound is immune stimulatory agent.

Also provided herein is a pharmaceutical composition comprising the LNP system or compositions components and a pharmaceutically acceptable carrier or excipient. In some aspects, the disclosure provides for a LNP formulation further comprising one or more pharmaceutical excipients. In some embodiments, the LNP formulation further comprises sucrose, tris, trehalose and/or glycine.

In some embodiments, any components of a composition are formulated with pharmaceutically acceptable excipients such as carriers, solvents, stabilizers, adjuvants, diluents, etc., depending upon the particular mode of administration and dosage form. In some embodiments, compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5.0 to about pH 8. In some embodiments, the composition has a therapeutically effective amount of at least one compound as described herein, together with one or more pharmaceutically acceptable excipients. Optionally, the composition can have a combination of the compounds described herein, or can include a second active ingredient useful in the treatment or prevention of bacterial growth (for example and without limitation, anti-bacterial or anti-microbial agents), or can include a combination of reagents of the disclosure.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

### Methods

As summarized above, the present disclosure provides methods, including methods using the herein described LNP systems and compositions. In some instances, methods of the present disclosure include co-administering components of a system that include an LNP-complexed payload DNA with an LNP-complexed auxiliary RNA to a subject in need thereof. For example, in some instances, a first LNP containing a first nucleic acid (e.g., an auxiliary RNA) is administered and a second LNP containing a second nucleic acid (e.g., a payload DNA) is administered. Where two different LNPs are employed, the different LNPs may be administered at essentially the same time or in series, e.g., the auxiliary RNA LNP may be administered followed by the payload DNA LNP, including where there is or is not time between administering the two LNPs. Any convenient method of co-administration, including those methods described herein, may be employed.

In some instances, methods of the present disclosure include administering a composition that includes an LNP co-formulation of payload DNA with auxiliary RNA to a subject in need thereof. Accordingly, in some instances, a single LNP that contains both auxiliary RNA and payload DNA may be administered.

The methods of the present disclosure provide various advantages, e.g., over conventional methods. For example, methods of the present disclosure may provide for the enhanced delivery of payload DNA to a desired cell or a desired location within a cell, for the enhanced expression from a payload DNA, for lessening an immune response to an LNP containing a payload DNA, or combinations of such advantages. In some instances, a method of the present disclosure may provide for both enhanced expression from a payload DNA of an LNP and a lessened immune response to the LNP. Advantages of the present methods, depending on the embodiment employed, may be present in any combination or singularly. Advantages of the present methods are not necessarily limited to those specifically described and, in some instances, the methods of the present disclosure, including methods using the systems and compositions described, may provide for other advantages.

In some instances, advantages of the herein described methods may include in an improvement in the delivery of a payload DNA to a desired location. In some instances, such an improvement is a 2-fold increase or more in the amount of payload DNA ultimately delivered to the nucleus relative to the amount of payload DNA that would be found in the nucleus if the delivery system did not include the auxiliary RNA, for example, a 2-fold, 3-fold, 4-fold, 5-fold, 6-fold, 7-fold, 8-fold, 10-fold, 15-fold, 20-fold, 30-fold, 40-fold, or 50-fold increase in the amount of payload DNA in the nucleus.

In some instances, the improvement is a 2-fold increase or more in the expression of an expression product of the payload DNA relative to the level of expression from the payload DNA that would be found if the delivery system did not include the auxiliary RNA, for example, a 2-fold, 3-fold, 4-fold, 5-fold, 6-fold, 7-fold, 8-fold, 10-fold, 15-fold, 20-fold, 30-fold, 40-fold, or 50-fold increase in the expression of an expression product of the payload DNA.

Numerous methods may be employed for assessing the delivery and of expression from a payload DNA. For example, in some instances, microscopy, e.g., with a nuclear counterstain (e.g., DAPI) may be employed to identify the presence of a fluorescent reporter encoded by the payload DNA in the nucleus of the cell. In some instances, other measurements of reporter expression, e.g., through measuring total fluorescence of a fluorescent reporter or in vivo bioluminescence from a bioluminescent reporter in an entire animal, in one or more tissues of an animal, or in cells or other biological samples collected from the animal, may be employed. In some instances, expression of a payload DNA may be assessed through measurement of the amount of expressed protein present in the blood or serum of an animal to which the LNP system has been administered (e.g., using an ELISA or other quantitative assay. In some instances, nucleic acid quantification methods, e.g., assessing copy number or the in situ location of certain nucleic acids, may be employed. Various quantitative and qualitative methods of measuring proteins and/or nucleic acids are readily available.

In some instances, the improvement is a decrease, e.g., a 1.5-fold or a 2-fold decrease or more in the immune response in a subject after administration of an LNP system that includes the payload DNA and auxiliary RNA relative to the level of the immune response that would be found if the delivery system did not include the auxiliary RNA, for example, a 1.5-fold, 2-fold, 3-fold, 4-fold, 5-fold, 6-fold, 7-fold, 8-fold, 10-fold, 15-fold, 20-fold, 30-fold, 40-fold, or 50-fold increase in the immune response in the subject. In some instances, measurement of one or more cytokine levels, including levels of expression and/or cytokine release, within the subject may be employed to evaluate the subject's immune response, including but not limited to e.g., the levels of IL-6 expression or cytokine release in the subject.

Numerous methods may be employed for assessing an immune reaction in a subject, e.g., after, including hours or days after, administration of LNP systems of the present disclosure. For example, in some instances, an immune response assessment may include measuring the level of one or more cytokines in the subject (e.g., by measuring the level of cytokine in a biological sample from the subject). Useful cytokines for evaluating an immune response include but are not limited to, e.g., IFNgamma, TNFalpha, Interleukin 1beta (IL-1beta), Interleukin 2 (IL-2), Interleukin 4 (IL-4), Interleukin 5 (IL-5), Interleukin 6 (IL-6), Interleukin 8 (IL-8), Interleukin 10 (IL-10), Interleukin 12p70 (IL-12p70), Interleukin 13 (IL-13), and the like. Various timepoints following administration may be assessed including but not limited to e.g., time points within ranges from 1 hr to 4 hr, from 2 hr to 6 hr, from 4 hr to 8 hr, from 6 hr to 12 hr, from 8 hr to 18 hr, from 12 hr to 24 hr, from 1 day to 2 days, from 2 days to 4 days, etc.

In some instances, methods of the present disclosure may include the use of systems for nuclear targeted delivery of the payload DNA. Targeting of a payload DNA to the nucleus of a cell may be achieved through a variety of approaches where such approaches may be used independently or in various combinations. In some instances, a NLS, or a portion thereof, may be included in or attached to a payload DNA. In some instances, a NLS, or a portion thereof, may be expressed from the auxiliary RNA and the payload DNA and expression product of the auxiliary RNA may be engineered to bind one another. For example, in some instances, a payload DNA may include a sequence to which a nuclear-targeting protein, such as a NTF, binds and the auxiliary RNA may include sequence encoding the NTF. Use of such mechanisms may be employed as a part of a DTS or in addition to an already present DTS.

In some embodiments, the NTF is active in the target cell and thus delivery of the DNA into the nucleus of the cell is facilitated without the need for additional exogenous factors. Accordingly, in some embodiments, methods for delivering payload DNA to the nucleus of a cell that utilize DTSs that leverage such already active DNA binding proteins may consist essentially of contacting the cell with one or more LNPs that include encapsulated payload DNA comprising the DTS and the auxiliary RNA.

As summarized above, methods of the present disclosure will generally involve the in vivo administration of LNPs, e.g., a system of LNP complexed auxiliary RNA and LNP complexed payload DNA or co-formulated LNPs that include both auxiliary RNA and payload DNA. In vivo administration of such systems and compositions may be accomplished through any suitable and effective means to any suitable subject in need thereof, including human and non-human, e.g., non-human primate, dog, rabbit, cat, horse, pig, rat, mouse, etc.

Systems and compositions of the present disclosure can also be administered directly to an organism for transduction of cells in vivo. Administration is by any of the routes normally used for introducing a molecule into ultimate contact with blood or tissue cells including, but not limited to, injection, infusion, topical application, and the like. Suitable methods of administering such compositions and systems are available and well known to those of skill in the art, and, although more than one route can be used to administer a particular composition, a particular route can often provide a more immediate and more effective reaction than another route.

Compositions and system components of the present disclosure may be administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition and system components may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, and other methods.

Any amount (dosage) can be used, which can be readily determined by the skilled artisan. In certain embodiments, the payload DNA of the administered LNP encodes a therapeutic and the dosage may be determined by evaluating the expression of the therapeutic, e.g., by measuring the concentration of the therapeutic in a blood or serum sample of a subject, and adjusting the dosage accordingly, e.g., to approach a known or suspected therapeutic dose or dose range for the therapeutic (e.g., know from other methods of delivery of the therapeutic, such as e.g., intravenous administration of recombinant therapeutic).

An effective amount of a subject compound will depend, at least, on the particular method of use, the subject being treated, the severity of the affliction, the manner of administration of the therapeutic composition, and the mechanism of action of the therapeutic. A “therapeutically effective amount” of a composition is a quantity of a specified compound sufficient to achieve a desired effect in a subject being treated.

Compositions as described herein can be administered to subjects by any suitable means and to any part, organ, or tissue of the subject. Non-limiting examples of administration means include portal vein infusion, umbilical vein infusion, direct splenic capsule injection, splenic artery infusion, and the like. In certain embodiments, compositions as described herein are administered directly to the liver (e.g., via portal vein injection) and/or via intra-splenic injection where the compositions will travel through the vasculature to reach the liver.

The instant methods may include the co-administration of one or more agents. The terms “co-administration” and “in combination with” include the administration of two or more agents either simultaneously, concurrently or sequentially within no specific time limits. In one embodiment, the agents are present in a solution, bodily fluid, target tissue, cell, cellular environment, and/or in a subject's body at the same time or exert their chemical, biological or therapeutic effect(s) at the same time. In one embodiment, the agents are in the same composition or unit dosage form. In other embodiments, the agents are in separate compositions or unit dosage forms. In certain embodiments, a first agent can be administered prior to (e.g., seconds, minutes, 5 minutes, 15 minutes, 30 minutes, 45 minutes, 1 hour, hours, 2 hours, 4 hours, 6 hours, or more, before), concomitantly with, or subsequent to (e.g., minutes, 5 minutes, 15 minutes, 30 minutes, 45 minutes, 1 hour, hours, 2 hours, 4 hours, 6 hours, or more, after) the administration of a second agent.

### Target Cells

Methods of the present disclosure are employed to deliver a payload DNA to a target cell, where the target cell may be one or more cell types of a subject. Accordingly, the target cells may vary and may include essentially any cell of a subject in need of delivery of the payload DNA or to which delivery of the payload DNA is otherwise desired.

Targeted cells include the cells of a targeted location, such as, e.g., the liver, or cells near or adjacent to hepatocytes, e.g., hepatocytes, hepatic stellate cells (HSCs), Kupffer cells (KCs), liver sinusoidal endothelial cells (LSECs), ductal cells, or combinations thereof.

It should be appreciated that the target cell may be of any origin, for example from an organism. In some embodiments, the target cell is a mammalian cell. Some non-limiting examples of a mammalian cell include, without limitation, a mouse cell, a rat cell, hamster cell, a rodent cell, and a nonhuman primate cell. In some embodiments, the target cell is a human cell. It should also be appreciated that the target cell may be of any cell type. For example, the target cell may be a stem cell, which may include embryonic stem cells, fetal stem cells, cord blood stem cells, or adult stem cells and progenitor cells (i.e., tissue specific stem cells, e.g., hematopoietic stem cells, mesenchymal stem or stromal cells, neural stem cells, etc.). In other cases, the target cell may be any differentiated cell type found in a subject. Cells of interest include both dividing cells and non-dividing cells. Examples of specific target cells of interest include, but are not limited to: hepatocytes, stellate cells, Kupffer cells (KCs), and liver sinusoidal endothelial cells, T lymphocytes, B lymphocytes, NK cells, skeletal muscle cells, cardiomyocytes, neurons, astrocytes, oligodendrocytes, dendritic cells, skin cells, photoreceptors, RPE cells, radial glia, etc.

### Treatment of Conditions

The systems, compositions, and methods of the present disclosure find use in treatment of a condition in a subject by administration of a therapeutically effective amount of an LNP composition, or the LNPs of a herein described system, to a subject in need thereof. As will be readily understood, the conditions that may be treated by use of the herein described systems, compositions, and methods will vary widely and will include essentially any genetic condition, e.g., where delivery of a nucleic acid to the cells of a subject allows for correction of a disease locus, replacement or supplementation of missing or insufficient expression of an necessary protein, silencing or repression of disease causing expression at the transcript or protein level, and the like. Conditions treated by the herein described methods need not necessarily be genetic conditions.

Conditions treatable by the herein described systems, compositions, and methods also include essentially any condition that may be treated by expression of a product from a delivered DNA, including e.g., diseases treatable by delivery of an effective amount of an oligonucleotide therapeutic (e.g., an antisense oligonucleotide, an siRNA, a microRNA, etc.), a polypeptide (e.g., therapeutic peptides and proteins such as peptide agonists and antagonists, protein agonists and antagonists, inc. endogenous and heterologous proteins, inc. antibodies and antibody fragments, etc.), and the like.

In some instances, the systems, compositions, and methods of the present disclosure allow for delivery of the payload DNA to the liver. Accordingly, subjects that may be treated include subjects having a liver condition as well as conditions other than those associated with the liver but treatable through expression of a product from a DNA in the liver, e.g., liver-related diseases.

For example, without limitation, a subject with an inherited condition, such as e.g., a monogenic disease, may be administered a gene therapy targeting the inherited condition through the methods as described herein. In some instances, a subject may be treated for a metabolic disease, including where the metabolic disease may or may not be an inherited condition. Treatment for a metabolic disease, according to the methods as described herein, may include gene therapy and/or delivery of a protein or peptide to treat the subject for the metabolic disease, e.g., through administration of a replacement enzyme or metabolite or the like.

In some instances, the condition for which a subject is treated is not a liver condition. Methods of the present disclosure may be employed to treat various conditions other than liver conditions. For example, the liver, or cells thereof, may be utilized for the expression of a cell extrinsic agent that treats a non-hepatic condition, e.g., in some embodiments an employed biologic may be a gene therapy agent that results in the expression of insulin or another hormone by liver cells to treat a diabetic condition or other endocrine disorder.

Examples of liver diseases and liver-related diseases include liver-related enzyme deficiencies, liver-related transport diseases, and the like. Such liver-related deficiencies may be acquired or inherited diseases and may include metabolic diseases (such as e.g., liver-based metabolic disorders). Inherited liver-based metabolic disorders may be referred to “inherited metabolic diseases of the liver”, such as but not limited to e.g., those diseases described in Ishak, Clin Liver Dis (2002) 6:455-479. Liver-related deficiencies may, in some instances, result in acute and/or chronic liver disease, including e.g., where acute and/or chronic liver disease is a result of the deficiency when left untreated or insufficiently treated.

Non-limiting examples of inherited liver-related enzyme deficiencies, liver-related transport diseases, and the like include Crigler-Najjar syndrome type 1; familial hypercholesterolemia, Factor VII deficiency, Glycogen storage disease type I, Glycogen storage disease type II (Pompe Disease), infantile Refsum's disease, Progressive familial intrahepatic cholestasis type 2, hereditary tyrosinemias (e.g., hereditary tyrosinemia type 1), genetic urea cycle defects, phenylketonuria (PKU), hereditary hemochromatosis, Alpha-I antitrypsin deficiency (AATD), Wilson Disease, and the like. Non-limiting examples of inherited metabolic diseases of the liver, including metabolic diseases having at least some liver phenotype, pathology, and/or liver-related symptom(s), include 5-beta-reductase deficiency, AACT deficiency, Aarskog syndrome, abetalipoproteinemia, adrenal leukodystrophy, Alpers disease, Alpers syndrome, alpha-1-antitrypsin deficiency, antithrombin III deficiency, arginase deficiency, argininosuccinic aciduria, arteriohepatic dysplasia, autoimmune lymphoproliferative syndrome, benign recurrent cholestasis, beta-thalassemia, Bloom syndrome, Budd-Chiari syndrome, carbohydrate-deficient glycoprotein syndrome, ceramidase deficiency, ceroid lipofuscinosis, cholesterol ester storage disease, cholesteryl ester storage disease, chronic granulomatous, chronic hepatitis C, Crigler-Najjar syndrome, cystic fibrosis, cystinosis, diabetes mellitus, Dubin-Johnson syndrome, endemic Tyrolean cirrhosis, erythropoietic protoporphyria, Fabry disease, familial hypercholesterolemia, familial steatohepatitis, fibrinogen storage disease, galactosemia, gangliosidosis, Gaucher disease, genetic hemochromatosis, glycogenosis type 1a, glycogenosis type 2, glycogenosis type 3, glycogenosis type 4, granulomatous disease, hepatic familial amyloidosis, hereditary fructose intolerance, hereditary spherocytosis, Hermansky-Pudlak syndrome, homocystinuria, hyperoxaluria, hypobetalipoproteinemia, hypofibrinogenemia, intrahepatic cholestasis of pregnancy, Lafora disease, lipoamide dehydrogenase deficiency, lipoprotein disorders, Mauriac syndrome, metachromatic leukodystrophy, mitochondrial cytopathies, Navajo neurohepatopathy, Niemann-Pick disease, nonsyndromic paucity of bile ducts, North American Indian childhood cirrhosis, ornithine transcarbamylase deficiency, partial lipodystrophy, Pearson syndrome, porphyria cutanea tarda, progressive familial intrahepatic cholestasis, progressive familial intrahepatic cholestasis type 1, progressive familial intrahepatic cholestasis type 2, protein C deficiency, Shwachman syndrome, Tangier disease, thrombocytopenia purpura, total lipodystrophy, type 1 glycogenosis, Tyrolean cirrhosis, tyrosinemia, urea cycle disorders, venocclusive disease, Wilson disease, Wolman disease, X-linked hyper-IgM syndrome, and Zellweger syndrome.

Conditions that may be treated using methods of the present disclosure will vary widely and are not limited to inherited liver-related enzyme deficiencies, liver-related transport diseases, or those specifically recited.

### Methods of Making

Any convenient methods can be used to prepare the LNPs of this disclosure. The LNP compositions can be prepared by high energy mixing of ethanolic lipids with aqueous DNA at low pH which protonates the ionizable lipid and provides favorable energetics for DNA/lipid association and nucleation of particles. The particles can be further stabilized through aqueous dilution and removal of the organic solvent. The particles can be concentrated to the desired level.

In some embodiments, LNPs may be formulated by mixing an organic solution of lipids with an aqueous solution of nucleic acid, including e.g., where the solution of nucleic acid includes, e.g., DNA only, RNA (e.g., mRNA) only, or a mixture of both DNA and RNA (e.g., mRNA) mixtures. For example, in some instances, separate DNA and RNA LNPs are prepared, e.g., by separately mixing an organic solution of lipids with an aqueous solution of DNA and separately mixing an organic solution of lipids with an aqueous solution of RNA. In some instances, DNA and RNA may be co-formulated by mixing an organic solution of lipids with an aqueous solution of DNA and RNA.

With respect to organic solutions of lipids, in some instances, lipidic excipient mixtures (e.g., ionizable lipid, helper lipid, cholesterol, PEG-lipid and potentially other targeting moieties) are dissolved in an organic solvent. With respect to aqueous solutions of the nucleic acid, such solutions may be prepared in a low pH buffer, e.g., buffer with a range of pH 3.0-4.0. In some instances, the lipid mixture is mixed with the aqueous nucleic acid solution at a flow ratio, where suitable flow ratios will vary and may include, e.g., a flow ratio of 1:3 (V/V). Mixing may be performed using a commercially available mixer device. Resulting solutions from mixing may be diluted with a buffer, such as e.g., a buffer with a pH range of 5.0-6.5.

Prepared LNP may be subjected to dialysis purification. For example, dialysis purification may be performed against a secondary buffer, including e.g., a secondary buffer with the pH range of 7.0-8.0. LNP solutions may be concentrated by an convenient method, including e.g., centrifugation, filtration, the like, and combinations thereof. The resulting LNPs may be assessed before use, according to any suitable method, including, but not limited to e.g., light scattering, RiboGreen assays, and the like. Such assays may be performed to determine e.g., particle size, encapsulation efficiency, and the like.

### Kits

Aspects of the present disclosure also include kits. Some aspects of this disclosure provide kits that include a LNP, one or more LNP compositions, LNP components, nucleic acid components, a payload DNA, an auxiliary RNA, e.g., as described above. Kits of the invention may further include one or more additional components, as described above, etc. In some instances, the various components of a given kit may be combined into a single composition, e.g., a composition comprising a single species of LNP, a composition comprising multiple species of LNP, as a pharmaceutical composition, etc., such as described above.

In some instances, kits may include an article of manufacture containing materials useful for the treatment of the disease, using the components described herein. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the present disclosure or an expression product of a compound of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. In some instances, the article of manufacture may contain an additional container comprising one or more additional components of the system, e.g., for co-administration with the primary component, e.g., an additional LNP comprising the auxiliary RNA of co-administration with the LNP comprising the payload DNA. In some instances, multiple components may be mixed before delivery. In some instances, components are provided in a ready-to-use format, e.g., that does not require mixing. Kits may further include other materials desirable from a commercial and user standpoint, including other buffers, diluents, filters, needles, syringes, and package inserts with instructions for use.

Components of the kits may be present in separate containers, or multiple components may be present in a single container. In addition to the above-mentioned components, a subject kit may further include instructions for using the components of the kit, e.g., to practice the subject methods. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium. In yet other embodiments, the actual instructions are not present in the kit, but means for obtaining the instructions from a remote source, e.g., via the internet, are provided. An example of this embodiment is a kit that includes a web address, provided as text or a scannable code, such as a bar code or QR code, where the instructions can be viewed and/or from which the instructions can be downloaded. As with the instructions, this means for obtaining the instructions is recorded on a suitable substrate.

### UTILITY

The subject methods, systems, and compositions, e.g., as described above, can be used in any application where in vivo delivery of a payload DNA is desired. Applications of interest include both research and therapeutic applications. Applications of interest include, but are not limited to: research applications, diagnostic applications, and therapeutic applications. In some instances, payload DNA that may be introduced via methods of the present disclosure include those methods where expression of the payload DNA of research proteins, diagnostic proteins, or therapeutic proteins is desired.

Research proteins are proteins whose activity finds use in a research protocol. As such, research proteins are proteins that are employed in an experimental procedure. The research protein may be any protein that has such utility, where in some instances the research protein is a protein domain that is also provided in research protocols by expressing it in a cell from an encoding nucleic acid. Examples of specific types of research proteins include, but are not limited to: reporters (including e.g., fluorescent, bioluminescent, and enzymatic reporters), transcription modulators of inducible expression systems, members of signal production systems, e.g., enzymes and substrates thereof, hormones, prohormones, proteases, enzyme activity modulators, peptide aptamers, antibodies, modulators of protein-protein interactions, genomic modification proteins, such as CRE recombinase, meganucleases, Zinc-finger nucleases, CRISPR/Cas-9 nuclease, TAL effector nucleases, etc., cellular reprogramming proteins, such as Oct 3/4, Sox2, Klf4, c-Myc, Nanog, Lin-28, etc., and the like.

Diagnostic proteins are proteins whose activity finds use in a diagnostic protocol. As such, diagnostic proteins are proteins that are employed in a diagnostic procedure. The diagnostic protein may be any protein that has such utility. Examples of specific types of diagnostic proteins include, but are not limited to: members of signal production systems, e.g., enzymes and substrates thereof, labeled binding members, e.g., labeled antibodies and binding fragments thereof, peptide aptamers, and the like.

Proteins of interest further include therapeutic proteins. Therapeutic proteins are proteins that provide a therapeutic benefit to a patient, and include secreted proteins, transmembrane proteins, and intracellularly acting proteins. As will be appreciated by one of ordinary skill in the art, payload DNAs that encode any protein that is associated with a liver disease, associated with a monogenic disease with a primary origin in the liver, or that, upon secretion from the liver, find use in treating one or more other organs or tissues in the body, may be delivered using the subject compositions and methods.

In some instances, the application of interest is a therapeutic application, for example, in the treatment of a disease. For example, the systems, compositions, and methods of the present disclosure may be used to deliver nucleic acid sequences to a cell to complement a genetic deficiency. As one nonlimiting example, compositions of the present application may be used in the treatment of a genetic deficiency that impacts the function of hepatocytes, or in the treatment of a genetic deficiency elsewhere in the body that can be remedied by leveraging hepatocytes as a biofactory to secrete a deficient protein or other DNA encoded therapeutic, such as e.g., a therapeutic RNA or polypeptide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

The following methods are applicable to the following examples.

LNP formulation. LNPs encapsulating nucleic acid payloads were prepared by mixing an organic solution of lipids with an aqueous solution of nucleic acid (e.g., DNA only, mRNA only, or DNA/mRNA mixtures). Briefly, the lipidic excipients mixture (ionizable lipid, helper lipid, cholesterol, PEG-lipid and potentially other targeting moieties) is dissolved in an organic solvent. An aqueous solution of the nucleic acid is prepared in a low pH buffer of range pH 3.0-4.0. The lipid mixture is then mixed with the aqueous nucleic acid solution at a flow ratio of 1:3 (V/V) using a commercially available mixer device. The resulting solution is immediately diluted with a buffer pH range of 5.0-6.5. The diluted LNP is subjected to dialysis purification against a secondary buffer with the pH range of 7.0-8.0. LNP solution is concentrated by using 100,000 MWCO Amicon Ultra centrifuge tubes (Millipore Sigma) followed by filtration through 0.2 μm PES sterilizing-grade filter. Particle size is determined by dynamic light scattering (Horiba nanoPartica SZ-100). Encapsulation efficiency is calculated by using Quant-it RiboGreen assay kit.

The following methods are applicable to the following examples.

EPO and cytokine detection in serum. Blood is collected via a retro-orbital bleed into serum separator tubes and processed to serum. The serum samples may be stored at −80 deg. C from collection until analysis. The serum levels of human EPO protein driven by expression from the payload DNA are quantified using the U-PLEX Human EPO Assay from MSD according to the manufacturer's instructions. The serum levels of mouse cytokines resulting from exposure to DNA-LNPs were quantified using the Mouse ProInflammatory 7-Plex Tissue Culture Kit from MSD according to the manufacturer's instructions.

### Example 1

Results—Expression from DNA LNPs is Depressed at Higher Doses In Vivo, which can be Improved by Co-Administration of LNPs Comprising a Stuffer DNA

LNPs formulated with EPO-encoding DNA were administered intravenously to wild-type mice at doses of 0.1 mg/kg (mpk), 0.3 mg/kg, and 1 mg/kg (FIG. 1) and EPO serum concentrations measured three days later. As shown in FIG. 1, increasing dosage resulted in increasing levels of EPO expression in a manner that was not dose-proportional. For example, serum EPO concentrations in mice that received 0.3 mg/kg were considerably less than 30% of the serum EPO concentrations in mice that received the 1 mg/kg dose.

The mechanism underlying this lack of dose proportionality was investigated further by assessing the serum levels of EPO achieved by simultaneously co-administering LNPs formulated with an EPO-encoding DNA and LNPs formulated with a GFP-encoding “stuffer” DNA. Serum EPO concentrations in mice dosed with 1 mpk, 0.3 mpk or 0.1 mpk of EPO DNA-LNP alone were substantially similar to those observed previously (FIG. 2, samples 1, 2 and 5 respectively—compare to FIG. 1). By contrast, statistically higher EPO serum concentrations—now proportional to the amount of EPO-encoding DNA delivered—were achieved in mice receiving the GFP-encoding stuffer DNA LNPs co-administered with EPO-encoding DNA LNPs (FIG. 2, compare sample 2 vs 3; compare sample 5 vs 6). This suggests the presence of a “sink” in the body that can be saturated by additional DNA material in the system, either delivered “in trans” i.e. as a distinct LNP formulation, e.g. as in FIG. 2 or “in cis”, i.e. within the same formulation, e.g. as in FIG. 1.

To determine if the sink would be saturated even more by delivering the stuffer DNA LNPs first, the same co-administration experiment was performed except that the LNPs formulated with the GFP-encoding DNA were dosed 15 minutes before dosing with LNPs formulated with the EPO-encoding DNA. Comparable EPO serum concentrations were achieved from this pre-dosing regimen as were achieved with simultaneous co-dosing (FIG. 2, compare sample 3 [EPO 0.3 mpk, GFP 0.7 mpk] vs 4 [EPO 0.3 mpk, GFP 0.7 mpk pre-dosed]).

Collectively and without being bound by any particular theory, these data demonstrate the presence of a mechanism which results in levels of expression from payload nucleic acids of LNPs dosed at lower levels that are substantially below that which would be expected from the expression observed when the same LNP is dosed at higher levels. It is noted that the amount of protein resulting from an LNP-delivered payload nucleic acid in an in vivo system is dependent on various factors, including not only the expression efficiency of the delivered payload but also the efficiency of cellular delivery and internalization, among other factors. These data further demonstrate that the effects of this mechanism can be attenuated through co-dosing and pre-dosing with LNPs containing non-therapeutic payloads to increase the expression from LNPs containing therapeutic payloads. The ability to interfere with the mechanism in this manner using nucleic acids of a different sequence implies a saturable mechanism that is non-specific (e.g., sequence independent).

### Example 2

Results—Co-Formulation of Auxiliary RNA with DNA in LNPs Results in Increased Expression from Payload DNAs and Reduced Immune Response

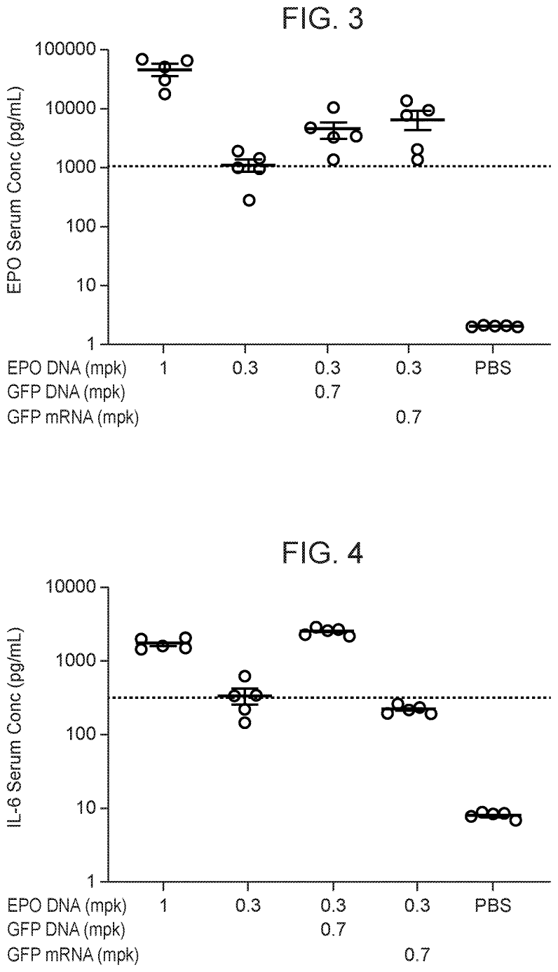

To confirm that the effect of stuffer DNA could be conveyed in cis (i.e. co-formulated with therapeutic DNA in LNPs), expression from an EPO-encoding payload DNA was measured as a function of serum EPO concentration in mice at seven days following administration of LNPs formulated with EPO-encoding DNA (“EPO DNA”) (FIG. 3, samples 1 and 2) versus LNPs co-formulated with EPO DNA and DNA encoding GFP (“GFP DNA”) (FIG. 3, sample 3). As observed in prior experiments, LNPs formulated with just EPO DNA and administered on their own at 0.3 mg/kg (mpk) or at 1 mg/kg resulted in a dose-disproportionately lower concentration of serum EPO at the 0.3 mpk dose versus the 1 mpk dose. LNPs co-formulated with 0.3 mpk EPO DNA plus 0.7 mpk of GFP DNA yielded considerably higher serum EPO concentrations versus formulations containing 0.3mpk EPO DNA alone. These higher concentrations were comparable to those achieved in prior co-dosing experiments, suggesting that the effect of stuffer DNA could indeed be conveyed in cis as well as in trans.

To determine if the effect of stuffer DNA could be achieved with any type of supplemental nucleic acid material, the effects of auxiliary RNA on in vivo expression from LNP-formulated payload DNAs were investigated by assessing the impact of RNA delivered in cis (i.e. co-formulated with therapeutic DNA in LNPs). Expression from an EPO-encoding payload DNA was measured as a function of serum EPO concentration in mice at seven days following administration of LNPs co-formulated with EPO DNA and mRNA encoding GFP (“GFP mRNA”) (FIG. 3, sample 4). Interestingly, LNPs co-formulated with 0.3 mpk EPO DNA plus 0.7 mpk of GFP mRNA also yielded considerably higher serum EPO concentrations versus formulations containing 0.3 mpk EPO DNA alone, which were comparable to EPO-DNA/GFP-DNA LNP coformulations. This suggests that an auxiliary RNA coformulated in the LNP with payload DNA will enhance expression at least as well as stuffer DNA.

Potency is only one side of the therapeutic coin. For a therapy to be efficacious it must not only be potent, but also be safe at that dose. Immune response activity, as measured by IL-6 cytokine release, was also assessed in wild-type mice following delivery of the various LNP test articles (FIG. 4). As expected, IL-6 serum concentrations in mice administered LNPs consisting of 0.3 mpk EPO DNA alone were lower than those of mice administered LNPs consisting of 1 mpk EPO DNA alone (FIG. 4, sample 2 versus 1). Mice administered LNPs co-formulated with 0.3 mpk EPO DNA and 0.7 mpk GFP DNA (essentially equivalent to 1 mpk total DNA) elicited serum IL-6 concentrations similar to the levels measured in mice administered LNPs formulated with 1 mpk EPO DNA alone (FIG. 4, sample 3). Surprisingly, mice administered LNPs co-formulated with 0.3 mpk EPO DNA and 0.7 mpk GFP mRNA elicited serum IL-6 concentrations comparable to the 0.3 mpk EPO DNA alone rather than the 1 mpk DNA dose level, be it as 1 mpk EPO DNA or 0.3 mpk EPO-DNA/0.7 mpk GFP DNA (FIG. 4, sample 4). The levels of IL-6 cytokine release in mice administered PBS negative control are provided as a baseline reference (FIG. 4, sample 5). Moreover, further studies have demonstrated that the observed effects of the auxiliary RNA are independent of whether the auxiliary RNA does or does not encode a protein as well as which protein is encoded if the auxiliary RNA does, in fact, encode a protein or portion thereof.

Collectively, these data demonstrate that LNP co-formulations of payload DNA of interest (e.g., EPO) together with stuffer DNA or RNA (e.g., GFP) result in levels of expression product from the therapeutic payload DNA that are higher than the levels observed in mice administered LNP formulations containing the corresponding amount of therapeutic payload DNA only (i.e., without the non-therapeutic payload). Notably, while the co-formulation of EPO DNA and GFP DNA did result in higher levels of EPO expression than the DNA alone formulation containing the same amount of EPO DNA, this DNA+DNA co-formulation resulted in a comparable immune response to that elicited by LNPs loaded with a same total amount of EPO DNA. In contrast, DNA and RNA LNP co-formulations, when administered to mice, resulted in lower levels of immune activation than corresponding DNA-only LNP formulations that contained similar amounts of total nucleic acid. As such, these findings demonstrate that, surprisingly, the addition of non-therapeutic payload RNA to therapeutic payload DNA-containing LNPs results in both higher expression (than administering a corresponding amount of payload DNA without additional RNA) and a reduced immune response (as compared to corresponding DNA-only LNPs with the same total nucleic acid dose) in vivo.

### Example 3

Results—DNA LNPs Co-Formulated with Auxiliary RNA Encoding a Nuclear Translocation Protein Also Demonstrates Enhanced Expression from Payload DNAs

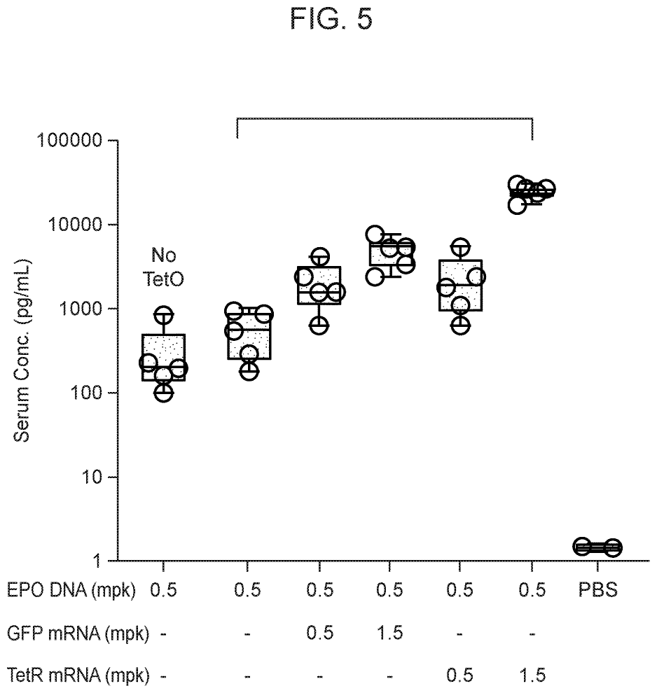

To confirm that the GFP encoded by the stuffer DNA or auxiliary mRNA was not the source of the improvement in potency, the efficacy of an LNP coformulated with a therapeutic DNA and an auxiliary RNA that encodes a protein that promotes nuclear translocation of DNA was investigated. Specifically, LNPs were formulated with an EPO DNA alone or with GFP mRNA or with an mRNA encoding the Tet repressor (TetR) protein (“TetR mRNA”). Except where indicated (i.e., “No TetO”), the EPO DNA constructs employed included tetracycline operator (TetO) sequences which are bound by TetR to promote nuclear translocation. Mice were dosed with LNP as indicated in FIG. 5 and serum EPO concentrations were measured three days following dosing.

As shown in FIG. 5, consistent with the above examples, LNP co-formulation of EPO DNA with an equal amount of GFP mRNA or TetR mRNA resulted in an increase in serum EPO as compared to LNP formulated with EPO DNA alone (compare e.g., sample 3 or sample 5 with sample 2). These findings show that the effect of enhanced expression observed with mRNA-DNA LNP co-formulations is independent of the coding sequence of the mRNA.

LNP co-formulation of EPO DNA with GFP mRNA at a higher mRNA to DNA ratio (i.e., sample 4, 1.5 mpk GFP mRNA+0.5 mpk EPO DNA; 3:1 ratio) resulted in a further increase in EPO expression as compared to EPO DNA co-formulated with GFP mRNA at a 1:1 ratio, indicating that more mRNA will further enhance potency.

EPO expression in mice administered LNPs co-formulated with a 3:1 TetR mRNA to EPO DNA ratio (0.5 mpk EPO DNA and 1.5 mpk TetR mRNA) was more than 2x higher than mice administered EPO DNA co-formulated with GFP mRNA at a corresponding ratio, indicating the contribution of TetR protein to enhancing expression through enhanced nuclear import of DNA. These data demonstrate that additional functions can be engineered into auxiliary RNAs that are co-formulated, co-administered, or pre-dosed with payload DNA LNPs. In this example, the TetR mRNA co-formulated LNPs provide not only the auxiliary RNA functions demonstrated with GFP mRNA co-formulations, but also enhanced expression through an added nuclear translocation function facilitated by the TetR and TetO components of the system. Note that while this example shows the coupling of the nuclear translocation with enhanced expression from auxiliary RNA co-formulation, the additional functions that can be incorporated into an auxiliary RNA are not limited to nuclear translocation.

Collectively, this example demonstrates that LNPs with payload DNAs co-formulated with auxiliary RNAs or co-administered with auxiliary RNA LNPs display enhanced expression of encoded payload DNAs in vivo. Moreover, such enhanced expression is independent of what, if any, gene product is encoded by the auxiliary RNA. Accordingly, additional functions can be engineered into auxiliary RNAs, including where those functions may be configured to further enhance the delivery and/or expression of the payload DNA.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A lipid nanoparticle (LNP) system comprising:
a payload DNA; and
an auxiliary RNA.

2. The LNP system of claim 1, wherein:
(i) the w/w ratio of auxiliary RNA to payload DNA is 1:1 or greater;
(ii) the payload DNA is at least 100 nucleotides in length;
(iii) the payload DNA is plasmid DNA (pDNA), nanoplasmid DNA (npDNA), doggy-bone DNA (dbDNA), mini-circle DNA (mcDNA), single-stranded circular DNA (circDNA), closed end DNA (ceDNA), or 3D DNA;
(iv) the auxiliary RNA is at least 30 nucleotides in length; or
(v) any combination selected from the group consisting of (i), (ii), (iii), and (iv).

3. The LNP system of claim 1 or 2, wherein the payload DNA comprises a coding sequence.

4. The LNP system of claim 3, wherein the coding sequence encodes a therapeutic polypeptide.

5. The LNP system of any of claim 3 or 4, wherein the payload DNA comprises an expression cassette comprising the coding sequence.

6. The LNP system of any of claims 1 to 5, wherein the payload DNA comprises a donor template.

7. The LNP system of any of claims 1 to 6, wherein the auxiliary RNA encodes a polypeptide.

8. The LNP system of claim 7, wherein the RNA-encoded polypeptide is a non-therapeutic polypeptide.

9. The LNP system of any of claims 1 to 8, wherein the auxiliary RNA is an mRNA.

10. The LNP system of any of claims 7 to 9, wherein the RNA-encoded polypeptide comprises a DNA binding domain (DBD) and the payload DNA comprises a sequence to which the DNA binding domain binds.

11. The LNP system of claim 10, wherein the DBD comprises a TetR DBD and the payload DNA comprises a TetO sequence.

12. The LNP system of claim 10, wherein the eDBD comprises a GAL4 DBD and the payload DNA comprises a UAS GAL4 binding site sequence.

13. The LNP system of any of claims 1 to 6, wherein the auxiliary RNA does not encode a polypeptide.

14. The LNP system of any of claims 1 to 13, wherein the auxiliary RNA comprises a noncoding RNA (ncRNA) sequence, optionally wherein at least 10%, 20%, 30%, 40%, 50%, 60%, 70%, 80%, 90%, or 100% of the auxiliary RNA comprises ncRNA sequence.

15. The LNP system of any of claims 1 to 14, wherein the auxiliary RNA comprises a synthetic RNA sequence, optionally wherein at least 10% of the auxiliary RNA comprises synthetic RNA sequence.

16. The LNP system of any of claims 1 to 15, wherein the auxiliary RNA comprises at least one synthetic nucleoside, a synthetic cap, or a combination thereof.

17. The LNP system of any of claims 1 to 16, wherein the payload DNA and auxiliary RNA are formulated within the same LNP particle.

18. The LNP system of any of claims 1 to 16, wherein the payload DNA and auxiliary RNA are formulated in separate LNP particles.

19. A lipid nanoparticle composition comprising a lipid nanoparticle complexed payload DNA and a lipid nanoparticle complexed auxiliary RNA.

20. The composition of claim 19, wherein:
(i) the w/w ratio of auxiliary RNA to payload DNA is 1:1 or greater;
(ii) the payload DNA is at least 100 nucleotides in length;
(iii) the payload DNA is plasmid DNA (pDNA), nanoplasmid DNA (npDNA), doggy-bone DNA (dbDNA), mini-circle DNA (mcDNA), single-stranded circular DNA (circDNA), closed ended DNA (ceDNA), or 3D DNA;
(iv) the auxiliary RNA is at least 30 nucleotides in length; or
(v) any combination selected from the group consisting of (i), (ii), (iii), and (iv).

21. The composition of claim 19 or 20, wherein the payload DNA and auxiliary RNA are complexed within the same LNP particle.

22. The composition of claim 19 or 20, wherein the payload DNA and auxiliary RNA are complexed within separate LNP particles.

23. The composition of any of claims 19 to 22, wherein the payload DNA comprises a coding sequence.

24. The composition of claim 23, wherein the coding sequence encodes a therapeutic polypeptide.

25. The composition of any of claim 23 or 24, wherein the payload DNA comprises an expression cassette comprising the coding sequence.

26. The composition of any of claims 19 to 25, wherein the payload DNA comprises a donor template.

27. The composition of any of claims 19 to 26, wherein the auxiliary RNA encodes a polypeptide.

28. The composition of claim 27, wherein the polypeptide is a non-therapeutic polypeptide.

29. The composition of any of claims 19 to 28, wherein the auxiliary RNA is an mRNA.

30. The composition of any of claims 27 to 29, wherein the encoded polypeptide comprises a DNA binding domain (DBD) and the payload DNA comprises a sequence to which the DNA binding domain binds.

31. The composition of claim 30, wherein the DBD comprises a TetR DBD and the payload DNA comprises a TetO sequence.

32. The composition of claim 30, wherein the DBD comprises a GAL4 DBD and the payload DNA comprises a UAS GAL4 binding site sequence.

33. The composition of any of claims 19 to 26, wherein the auxiliary RNA does not encode a polypeptide.

34. The composition of any of claims 19 to 33, wherein the auxiliary RNA comprises a noncoding RNA (ncRNA) sequence, optionally wherein at least 10% of the auxiliary RNA comprises ncRNA sequence.

35. The composition of any of claims 19 to 34, wherein the auxiliary RNA comprises a synthetic RNA sequence, optionally wherein at least 10% of the auxiliary RNA comprises synthetic RNA sequence.

36. The composition of any of claims 19 to 35, wherein the auxiliary RNA comprises at least one synthetic nucleoside, a synthetic cap, or a combination thereof.

37. The composition of any of claims 19 to 36, wherein the composition further comprises a pharmaceutically acceptable carrier or excipient.

38. A method comprising:
administering to a subject an amount of a lipid nanoparticle (LNP) system, the LNP system comprising:
a payload DNA encoding an expression product; and
an auxiliary RNA,
sufficient to cause cells of the subject to express the expression product, wherein:
(a) the expression product is expressed at a level at least twice as high as the level of expression when the subject is administered a corresponding LNP system that comprises the payload DNA but does not comprise the auxiliary RNA;
(b) the subject produces a reduced immune response as compared to administration of a corresponding LNP system does not comprise the auxiliary RNA but results in an equivalent level of the expression product; or
(c) both (a) and (b).

39. The method of claim 38, wherein the payload DNA and auxiliary RNA are formulated in separate LNP particles.

40. The method of claim 39, wherein the auxiliary RNA LNP is administered before the payload DNA LNP.

41. The method of claim 39, wherein the auxiliary RNA LNP and the payload DNA LNP are co-administered.

42. The method of claim 38, wherein the payload DNA and auxiliary RNA are co-formulated within the same LNP particle.

43. The method of any of claims 38 to 42, wherein:
(i) the w/w ratio of auxiliary RNA to payload DNA is 1:1 or greater;
(ii) the payload DNA is at least 100 nucleotides in length;
(iii) the payload DNA is plasmid DNA (pDNA), nanoplasmid DNA (npDNA), doggy-bone DNA (dbDNA), mini-circle DNA (mcDNA), single-stranded circular DNA (circDNA), closed ended DNA (ceDNA), or 3D DNA;
(iv) the auxiliary RNA is at least 30 nucleotides in length; or
(v) any combination selected from the group consisting of (i), (ii), (iii), and (iv).

44. The method of any of claims 38 to 43, wherein the payload DNA comprises a coding sequence.

45. The method of claim 44, wherein the coding sequence encodes a therapeutic polypeptide.

46. The method of any of claim 44 or 45, wherein the payload DNA comprises an expression cassette comprising the coding sequence.

47. The method of any of claims 38 to 46, wherein the payload DNA comprises a donor template.

48. The method of any of claims 38 to 47, wherein the auxiliary RNA encodes a polypeptide.

49. The method of claim 48, wherein the polypeptide is a non-therapeutic polypeptide.

50. The method of any of claims 38 to 49, wherein the auxiliary RNA is an mRNA.

51. The method of any of claims 48 to 50, wherein the RNA-encoded polypeptide comprises a DNA binding domain (DBD) and the payload DNA comprises a sequence to which the DNA binding domain binds.

52. The method of claim 51, wherein the DBD comprises a TetR DBD and the payload DNA comprises a TetO sequence.

53. The method of claim 51, wherein the DBD comprises a GAL4 DBD and the payload DNA comprises a UAS GAL4 binding site sequence.

54. The method of any of claims 38 to 47, wherein the auxiliary RNA does not encode a polypeptide.

55. The method of any of claims 38 to 54, wherein the auxiliary RNA comprises a noncoding RNA (ncRNA) sequence, optionally wherein at least 10% of the auxiliary RNA comprises ncRNA sequence.

56. The method of any of claims 38 to 55, wherein the auxiliary RNA comprises a synthetic RNA sequence, optionally wherein at least 10% of the auxiliary RNA comprises synthetic RNA sequence.

57. The method of any of claims 38 to 56, wherein the auxiliary RNA comprises at least one synthetic nucleoside, a synthetic cap, or a combination thereof.

58. The method of any of claims 38 to 57, further comprising assessing, in one or more biological samples collected from the subject after the administering: the level of expression of the expression product, the level of immune response, or both.

59. A kit comprising:
an auxiliary RNA, components sufficient for complexing the auxiliary RNA with a lipid nanoparticle (LNP) and, optionally, with a payload DNA, and optionally, the payload DNA;
a LNP complexed auxiliary RNA, components for complexing a payload DNA with a LNP, and optionally, the payload DNA; or
a LNP complexed auxiliary RNA and payload DNA;
optionally further comprising a pharmaceutically acceptable carrier or excipient suitable for administering LNP complexed auxiliary RNA and payload DNA to a subject in need thereof.

60. The kit of claim 59, wherein:
(i) the w/w ratio of auxiliary RNA to payload DNA is 1:1 or greater;
(ii) the payload DNA is at least 100 nucleotides in length;
(iii) the payload DNA is plasmid DNA (pDNA), nanoplasmid DNA (npDNA), doggy-bone DNA (dbDNA), mini-circle DNA (mcDNA), single-stranded circular DNA (circDNA), closed ended DNA (ceDNA), or 3D DNA;
(iv) the auxiliary RNA is at least 30 nucleotides in length; or
(v) any combination selected from the group consisting of (i), (ii), (iii), and (iv).

61. The kit of claim 59 or 60, wherein the payload DNA and auxiliary RNA are complexed within the same LNP particle.

62. The kit of claim 59 or 60, wherein the payload DNA and auxiliary RNA are complexed within separate LNP particles.

63. The kit of any of claims 59 to 62, wherein the payload DNA comprises a coding sequence.

64. The kit of claim 63, wherein the coding sequence encodes a therapeutic polypeptide.

65. The kit of any of claim 63 or 64, wherein the payload DNA comprises an expression cassette comprising the coding sequence.

66. The kit of any of claims 59 to 65, wherein the payload DNA comprises a donor template.

67. The kit of any of claims 59 to 66, wherein the auxiliary RNA encodes a polypeptide.

68. The kit of claim 67, wherein the polypeptide is a non-therapeutic polypeptide.

69. The kit of any of claims 59 to 68, wherein the auxiliary RNA is an mRNA.

70. The kit of any of claims 67 to 69, wherein the encoded polypeptide comprises a DNA binding domain (DBD) and the payload DNA comprises a sequence to which the DNA binding domain binds.

71. The kit of claim 70, wherein the DBD comprises a TetR DBD and the payload DNA comprises a TetO sequence.

72. The kit of claim 70, wherein the DBD comprises a GAL4 DBD and the payload DNA comprises a UAS GAL4 binding site sequence.

73. The kit of any of claims 59 to 66, wherein the auxiliary RNA does not encode a polypeptide.

74. The kit of any of claims 59 to 73, wherein the auxiliary RNA comprises a noncoding RNA (ncRNA) sequence, optionally wherein at least 10% of the auxiliary RNA comprises ncRNA sequence.

75. The kit of any of claims 59 to 74, wherein the auxiliary RNA comprises a synthetic RNA sequence, optionally wherein at least 10% of the auxiliary RNA comprises synthetic RNA sequence.

76. The kit of any of claims 59 to 75, wherein the auxiliary RNA comprises at least one synthetic nucleoside, a synthetic cap, or a combination thereof.

77. The kit of any of claims 59 to 76, wherein the kit further comprises a delivery device suitable for administration of LNP complexed auxiliary RNA and payload DNA to a subject, optionally wherein the device is an injection or infusion device.

78. A method of making a lipid nanoparticle (LNP) complexed auxiliary RNA and payload DNA, the method comprising:
mixing an organic solution of lipids with an aqueous solution of nucleic acid comprising a mixture of auxiliary RNA and payload DNA; or
mixing a first organic solution of lipids with a first aqueous solution of nucleic acid comprising auxiliary RNA and mixing a second organic solution of lipids with a second aqueous solution of nucleic acid comprising payload NDA.

79. The method of claim 78, wherein the auxiliary RNA and the payload DNA are present together in the mixture of auxiliary RNA and payload DNA at a w/w ratio of 1:1 or greater; or wherein the first and second aqueous solutions contain a w/w ration of auxiliary RNA to payload DNA of 1:1 or greater.

80. The method of claim 78 or 79, wherein:
(i) the payload DNA is at least 100 nucleotides in length;
(ii) the payload DNA is plasmid DNA (pDNA), nanoplasmid DNA (npDNA), doggy-bone DNA (dbDNA), mini-circle DNA (mcDNA), single-stranded circular DNA (circDNA), closed ended DNA (ceDNA), or 3D DNA;
(iii) the auxiliary RNA is at least 30 nucleotides in length; or
(iv) any combination selected from the group consisting of (i), (ii), and (iii).

81. The method of any of claims 78 to 80, wherein the payload DNA comprises a coding sequence.

82. The method of claim 81, wherein the coding sequence encodes a therapeutic polypeptide.

83. The method of any of claim 81 or 82, wherein the payload DNA comprises an expression cassette comprising the coding sequence.

84. The method of any of claims 78 to 83, wherein the payload DNA comprises a donor template.

85. The method of any of claims 78 to 84, wherein the auxiliary RNA encodes a polypeptide.

86. The Method of claim 85, wherein the polypeptide is a non-therapeutic polypeptide.

87. The method of any of claims 78 to 86, wherein the auxiliary RNA is an mRNA.

88. The method of any of claims 85 to 87, wherein the encoded polypeptide comprises a DNA binding domain (DBD) and the payload DNA comprises a sequence to which the DNA binding domain binds.

89. The method of claim 88, wherein the DBD comprises a TetR DBD and the payload DNA comprises a TetO sequence.

90. The method of claim 88, wherein the DBD comprises a GAL4 DBD and the payload DNA comprises a UAS GAL4 binding site sequence.

91. The method of any of claims 78 to 84, wherein the auxiliary RNA does not encode a polypeptide.

92. The method of any of claims 78 to 91, wherein the auxiliary RNA comprises a noncoding RNA (ncRNA) sequence, optionally wherein at least 10% of the auxiliary RNA comprises ncRNA sequence.

93. The method of any of claims 78 to 92, wherein the auxiliary RNA comprises a synthetic RNA sequence, optionally wherein at least 10% of the auxiliary RNA comprises synthetic RNA sequence.

94. The method of any of claims 78 to 93, wherein the auxiliary RNA comprises at least one synthetic nucleoside, a synthetic cap, or a combination thereof.

95. The method of any of claims 78 to 94, wherein the payload DNA and auxiliary RNA are formulated within the same LNP particle.

96. The method of any of claims 78 to 94, wherein the payload DNA and auxiliary RNA are formulated in separate LNP particles.
